(19) United States
(12) Patent Application Publication
(10) Pub. No.: US 2009/0175848 A1
(43) Pub. Date: Jul. 9, 2009

(54) Modulation of the Cooperativity Between the Ion Channels TRPM5 and TRPA1

(76) Inventors: S. Paul Lee, Newtown, PA (US); Tulu Buber, Newtown, PA (US); Rok Cerne, Lawrenceville, NJ (US); Robert W. Bryant, Princeton, NJ (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/212,508

(22) Filed: Sep. 17, 2008

Related U.S. Application Data
(60) Provisional application No. 60/973,080, filed on Sep. 17, 2007.

(51) Int. Cl. A61K 39/395; C12N 5/06; C12N 5/08; A61K 31/7105; A61K 31/7088; C12Q 1/68; C12Q 1/44

(52) U.S. Cl. 424/130.1; 435/375; 435/366; 514/44; 435/29; 435/19

(57) ABSTRACT

The present invention is related to modulating TRPA1 ion channel activity by targeting the ion channel TRPM5. The cooperativity between the ion channels can be used to modulate pain, mechanosensation and taste responses triggered through TRPA1 by modulating the activity of TRPM5

### CROSS REFERENCE TO RELATED APPLICATIONS

This application claims the benefit of U.S. Provisional Appl. No. 60/973,080, filed Sep. 17, 2007, which is herein incorporated by reference.

### BACKGROUND OF THE INVENTION

1. Field of the Invention

The present invention is related to modulating TRPA1 ion channel activity by targeting the ion channel TRPM5 and vice versa through the cooperativity mechanism identified herein. More specifically, the present invention relates to methods of modulating pain, mechanosensation and taste responses triggered through the ion channels.

2. Background

Ion channels are transmembrane proteins that form pores in a membrane and allow ions to pass from one side to the other (reviewed in B. Hille (Ed), 1992, Ionic Channels of Excitable Membranes 2nd ed., Sinauer, Sunderland, Mass.). Several ion channels have been shown to be essential for taste transduction (Perez et al., Nature Neuroscience 5: 1169-1176 (2002); Zhang et al., Cell 112: 293-301 (2003)). The effects that well known taste compounds have on ion channel activity have also begun to be analyzed. For example, menthol has been shown to activate the transient receptor potential (TRP) channel M8 (TRPM8) (Behrendt, H. -J., et al., Brit. J Pharm. 141: 737-745 (2004)).

The TRP channel A1 (TRPA1) is also a member of the superfamily of TRP channels. TRPA1 was initially described as a cold sensitive, nonselective cation channel (Story, G. M. et al., Cell 112: 819-829 (2003)), but it also functions as a ligand-gated channel in heterologous expression systems and sensory neurons. (Ramsey, I. S. et al., Ann. Rev. Physiol. 68: 619-647 (2006)). Noxious stimuli, including natural compounds such as cinnamaldehyde and the ingredients in mustard (allyl isothiocyanate, AITC), cold temperatures and environmental irritants all activate TRPA1 (Jordt, S. E., et al., Nature 427: 260-265 (2004); Macpherson, L. J., et al., Curr. Biol. 15: 929-934 (2005); Macpherson, L. J., et al., Nature 445: 541-545 (2007); Bautista, D. M. et al., Proc. Natl. Acad. Sci. USA 102: 12248-12252 (2005); Bandell, M., et al., Neuron 41: 849-857 (2004); Kwan, K. Y., et al., Neuron 50: 277-289 (2006)). TRPA1 has also been shown to be important in responses to pain. (Bautista, D. M. et al., Cell 124: 1269-1282 (2006); Trevisani et al. Proc. Natl. Acad. Sci. USA 104: 13519-13524 (2007)).

Recent studies have shown noxious stimuli activate TRPA1 through an unusual mechanism involving covalent modification of cysteine and lysine residues within the N-terminal cytoplasmic domain of the channel protein (Hinman, A., et al., Proc. Natl. Acad. Sci. USA 103: 19564-19568 (2006); Macpherson, L. J. et al. (2007)). In addition, one model suggests TRPA1 activation by bradykinin, a potent algogenic (pain related) substance released in response to tissue injury and inflammation, occurs through two possible mechanisms: (1) through PLC-mediated increases in intracellular Ca²⁺ or other metabolites; or (2) via Ca²⁺ influx through TRPV1 (Dorener, J. F. et al., J. Biol. Chem. 282: 13180-13189 (2007); Bautista, D. M. et al., (2006); Akopain et al. J. Physiol. 583: 175-193 (2007)).

Pain is a sensory experience distinct from sensations of touch, pressure, heat and cold. It is often described by sufferers by such terms as bright, dull, aching, pricking, cutting or burning and is generally considered to include both the original sensation and the reaction to that sensation. This range of sensations, as well as the variation in perception of pain by different individuals, renders a precise definition of pain difficult, however, many individuals suffer with severe and continuous pain.

TRPM5 is another member of the TRP superfamily. TRPM5 is believed to be activated by stimulation of a receptor pathway coupled to phospholipase C and by IP₃-mediated Ca²⁺ release. The opening of this channel is dependent on a rise in Ca²⁺ levels (Hoffmann et al., Current Biol. 13: 1153-1158 (2003)). TRPM5 is also a necessary part of the taste-perception machinery and has been shown to play a role in bitter, sweet and umami taste (Talavera, K. et al., Nature 438: 1022-1025 (2005)).

An earlier study that analyzed TRP channel distribution in mice demonstrated that TRPM5 expression is quite limited (Kunert-Keil et al. BMC Genomics, 7: 159 (2006)). This earlier study did not identify TRPM5 expression in nerve tissue or its association with pain.

Therefore, there exists a need in the art to provide a method to modulate the activity of these ion channels. The present invention identifies a cooperativity mechanism between TRPA1 and TRPM5. Identification of this mechanism allows for the specific modulation of the cognate channels through their common pathway. The common pathway also provides the basis for modulating their activity, especially with respect to modulating taste, mechanosensation and decreasing pain responses.

### BRIEF SUMMARY OF THE INVENTION

A new cooperativity between the ion channels TRPA1 and TRPM5 has been identified. The common pathway provides the basis for modulating their activity, especially with respect to modulating taste, mechanosensation and decreasing pain responses.

An embodiment of the invention is a method for modulating TRPA1-mediated processes comprising administering a modulator of TRPM5 activity. In one embodiment, the TRPA1 and TRPM5 are human. In another embodiment, the administration is done in vivo. In yet another embodiment, the TRPA1 is present in a TRPM5-expressing cell or cultured neuron. In a further embodiment, the modulated processes are selected from the group consisting of pain, mechanosensation and taste. The TPRM5 activities may be either increased or decreased.

In another embodiment, the invention relates to inhibiting TRPA1-mediated pain signaling by inhibiting TRPA1 activity, comprising administering to a subject in need thereof an inhibitor of TRPM5 expression. In one embodiment, the TRPA1 is present in a TRPM5-expressing cell or cultured neuron. In another embodiment, the TRPA1 and TRPM5 are human. In a further embodiment, TRPM5 expression is inhibited using RNA interference, antisense oligonucleotides, ribozymes, aptamers or antibodies. In yet another embodiment, the TRPA1 activity is measured by measuring calcium influx in said TRPA1-expressing cell or by measuring the enzymatic activity of the phospholipase C polypeptide. The enzymatic activity can be the breakdown of phosphatidylinositol-4,5-bisphospate (PIP2) into diacylglycerol (DAG) and inositol triphosphate (IP3). In another embodiment, the type of pain is selected from the group consisting of acute, chronic, neuropathic and nociceptive.

In another embodiment, the invention relates to a method of inhibiting TRPA1-mediated signaling comprising administering an inhibitor of TRPM5.

In another embodiment, the invention relates to a method of increasing TRPA1 expression in a cell comprising expressing TRPM5 in said cell. In one embodiment, TRPM5 expression is at a greater level than expressed in wild-type cells. In another embodiment, the TRPM5 is exogenously added to said TRPA1 expressing cell.

In another embodiment, the invention relates to a method of amplifying TRPM5 activation comprising administering an activator of TRPA1 activity. In one embodiment, the activator of TRPA1 is selected from the group consisting of cinnamaldehyde, eugenol, gingerol, methyl salicylate, AITC and allicin.

In another embodiment, the invention relates to a method of blocking TRPM5 activity comprising administering an inhibitor of TRPA1 activity.

In another embodiment, the invention relates to a method for identifying an agent that inhibits TRPA1 activity through TRPM5 signaling comprising: (a) contacting a cell that expresses both TRPA1 and TRPM5 with an agent; (b) measuring the activity of TRPM5, (c) contacting another cell that expresses both TRPA1 and TRPM5 with the same agent as in step (a); (d) measuring the activity of TRPA1; and (e) identifying an agent that decreases both TRPM5 and TRPA1 activity. In some embodiments, control cells in which a TRPM5 response cannot be generated, are used. In further embodiments, the control cells are chinese hamster ovary cells. In one embodiment, the TRPA1 and TRPM5 are human. In other embodiments, the TRPM5 activity is measured by measuring the membrane potential of said cell or by measuring calcium influx in said cell. In another embodiment, the TRPA1 activity is measured by measuring the enzymatic activity of phospholipase C, wherein the enzymatic activity can be the breakdown of phosphatidylinositol-4,5-bisphospate (PIP2) into diacylglycerol (DAG) and inositol triphosphate (IP3).

In another embodiment, the invention relates to a method of modulating calcium-activated ion channel activity comprising administering a modulator of TRPA1 activity to a cell. In one embodiment, the calcium-activated ion channel is TRPM5. In another embodiment, the modulator of TRPA1 activity is selected from the group consisting of cinnamaldehyde, eugenol, gingerol, methyl salicylate, AITC and allicin. In further embodiments, the calcium-activated ion channel activity is measured by measuring the membrane potential of said cell or by measuring calcium influx in said cell.

Further embodiments, features, and advantages of the present inventions, as well as the structure and operation of the various embodiments of the present invention, are described in detail below with reference to the accompanying drawings.

### BRIEF DESCRIPTION OF THE DRAWINGS/FIGURES

The accompanying drawings, which are incorporated herein and form a part of the specification, illustrate one or more embodiments of the present invention and, together with the description, further serve to explain the principles of the invention and to enable a person skilled in the pertinent art to make and use the invention.

FIG. 1 shows the ability of the TRPA1 agonist AITC to trigger a strong membrane potential response in TRPM5-expressing HEK-293 cells (TRPM5-293) based on FLIPR® traces using increasing concentrations of AITC.

FIG. 2 shows that AITC triggers Ca²⁺ influx only in TRPM5-293 cells based on FLIPR® traces using increasing concentrations of AITC. AITC has no effect on parental HEK-293 cells.

FIG. 3 shows that AITC causes a response in TRPM5-293 cells but not Chinese hamster ovary (CHO) cells expressing TRPM5.

FIG. 4 shows the electrophysiological response caused by AITC in TRPM5-293 cells.

FIG. 5 shows that the electrophysiological response by AITC on TRPM5-293 cells is voltage dependent.

FIG. 6 shows that not only does AITC trigger responses in TRPM5-293 cells, but close AITC analogs that are active on TRPA1 also activate TRPM5-293 cells.

FIG. 7 shows human TRPA1 si-RNA blocks the AITC response in TRPM5-293 cells based on FLIPR® traces.

FIG. 8 shows that expression of TRPM5 in TRPM5-293 cells strongly increases low, endogenous levels of TRPA1 present in the cells.

FIG. 9 shows that pre-incubation with EGTA alters the kinetics of the membrane potential traces generated by AITC in TRPM5-293 cells based on FLIPR® traces.

FIG. 10 shows that chelation of extracellular Ca²⁺ with EGTA blocks AITC-mediated calcium responses in TRPM5-293 cells based on FLIPR® traces.

FIG. 11 shows that the phophoslipase C (PLC) blocker U73122 enhances the membrane potential response of both AITC and ionomycin. Inhibition of PLC by U73122 and consequently the inhibition of an internal Ca²⁺ signal does not block the AITC-mediated change in the membrane potential.

FIG. 12 shows that U73122 enhances the calcium response of AITC.

FIG. 13 shows that the specific TRPM5 inhibitor LG 21589 blocks AITC membrane potential responses in TRPM5 transfected HEK cells. The 3 μM and 33 μM concentrations were chosen because these are the concentrations closest to the EC₅₀ and EC₉₀, respectively.

FIG. 14 shows that the specific TRPA1 inhibitor RPB-A1|1 (LG49628) blocks AITC membrane potential responses in TRPM5-293 cells and does not affect ATP responses in those cells, a heterologous ion channel or the effect of capsaicin on TRPV1-expressing HEK 293 cells.

FIG. 15 shows TRPM5 and TRPA1 expression in mouse dorsal ganglion primary cell culture and cDNA by PCR. Lane 1, mTRPM5 primer set+mouse dorsal ganglion primary cell culture cDNA; Lane 2, mTRPM5 primer set+mouse dorsal ganglion cDNA; Lane 3, mTRPM5 primer set+no template control; Lane 4, mTRPA1 primer set+mouse dorsal ganglion primary cell culture cDNA; Lane 5, mTRPA1 primer set+mouse dorsal ganglion cDNA; Lane 6, mTRPA1 primer set+no template control; Lane 7, 100 bp ladder.

FIG. 16 shows staining of LacZ-positive freshly isolated taste epithelial cells with fluorescein digalactoside. Taste cells isolated from a LacZ-TRPM5 mouse were positive for TRPM5 expression.

FIG. 17 shows staining of LacZ-positive freshly isolated dorsal root ganglion neurons with fluorescein digalactoside. Neuronal cells isolated from a LacZ-TRPM5 mouse were positive for TRPM5 expression.

### DETAILED DESCRIPTION OF THE INVENTION

### Overview

The present invention provides a method of modulating TRPA1 activity by targeting the TRPM5 ion channel and vice versa through the cooperativity mechanism identified herein. The present invention is predicated in part on the discovery that TRPA1 is modulated (activated or inhibited) by the TRPM5 ion channel. In accordance with these discoveries, the present invention provides methods of modulating TRPA1 activities and also methods of identifying TRPM5-specific modulators that effect TRPA1 activity. The present invention also provides methods for modulating calcium-activated ion channels (such as TRPM5) using modulators of TRPA1. The claimed invention also relates to therapeutic applications of such compounds.

As used in the specification and claims, the singular form “a”, “an” and “the” include plural references unless the context clearly dictates otherwise. For example, the term “an ion channel” includes a plurality of ion channels. The term “a cell” includes a plurality of cells.

As described above, human and mouse TRPA1 ion channels are activated by noxious cold temperatures. TRPA1 is also activated by an algogenic peptide and a variety of natural pungent compounds present in foods and flavoring products. Cinnamaldehyde, a specific TRPA1 activator in vitro, predominantly excites cold-sensitive DRG neurons in culture. The response profile of menthol and cinnamaldehyde accurately reflect the mutually exclusive expression of the two cold-activated ion channels TRPM8 and TRPA1, respectively. In addition, external Ca²⁺ has been shown to augment cold-induced activation of TRPA1 but is not required for cinnamaldehyde-induced activation. Therefore, as used herein, TRPA1-mediated processes include, but are not limited to pain, mechanosensation and taste.

As mentioned above, TRPA1 is activated by cinnamaldehyde and other sensory compounds. These include a variety of pungent compounds—allicin from fresh garlic, mustard, wintergreen, ginger, and clove, which all activate TRPA1. Cinnamaldehyde is the main constituent of cinnamon oil (˜70%) and is extensively used for flavoring purposes in foods, chewing gums, and toothpastes. AITC (mustard oil) is one of the active ingredients in horseradish and wasabi. Methyl Salicylate (wintergreen oil) is used commonly in products such as Listerine, IcyHot, and Bengay for its burning effect.

The claimed methods have various applications. By activating TRPA1, these compounds, e.g., allicin, eugenol, gingerol, methyl salicylate, AITC and cinnamaldehyde, can stimulate sensory perception by a subject. This could have many practical utilities. For example, modulating the activity of these compounds can be used to alter flavoring of various compositions or products, as well as blocking unfavorable tastes associated with these compounds.

By altering sensations, the TRPA1-modulating compounds can be used as food additives to either enhance or block flavors of various foodstuffs to which they are added. Flavoring agents, individually or in combination, are used to impart desired flavor characteristics to a variety of consumable products. The TRPA1-activating compounds of the present invention can be used alone or in combination with other flavoring agents in order to provide interesting and pleasing flavor perceptions.

Importantly, the ability of TRPA1 to modulate calcium-activated ion channels can be exploited to modulate other processes. For example, TRPM5 has been shown to be important in bitter taste sensations and to enhance the perception of sweet taste. Therefore, TRPA1 modulators can be used to modulate bitter and sweet tastes.

In addition to their use in the food industry, TRPA1-modulating compounds can also be used in other fields where enhanced sensory perception is desired. For example, the TRPA1-activating compounds can find applications in body-care or cosmetic products. In general, these compounds can be used in all fields in which a cooling effect is to be imparted to the products in which they are incorporated. By way of example one may cite beverages such as fruit juices, soft drinks or cold tea, ice creams and sorbets, sweets, confectioneries, chewing gum, chewing tobacco, cigarettes, pharmaceutical preparations, dental-care products such as dentifrice gels and pastes, mouth washes, gargles, body and hair care products such as shampoos, shower or bath gels, body deodorants and antiperspirants, after-shave lotions and balms, shaving foams, perfumes, etc.

In addition to the above-noted uses, since TRPA1 is activated by the algogenic inflammatory peptide bradykinin (BK), an important use for the present invention is in the management of pain. The activation of many TRP ion channels is linked to G protein coupled receptor (GPCR) signaling. BK directly excites nociceptive DRG neurons and causes hyperalgesia.

“Pain” is a sensory experience perceived by nerve tissue distinct from sensations of touch, pressure, heat and cold. The range of pain sensations, as well as the variation of perception of pain by individuals, renders a precise definition of pain near impossible. In the context of the present invention, “pain” is used in the broadest possible sense and includes nociceptive pain, such as pain related to tissue damage and inflammation, pain related to noxious stimuli, acute pain, chronic pain, and neuropathic pain.

Pain that is caused by damage to neural structures is often manifest as a neural supersensitivity or hyperalgesia and is termed “neuropathic” pain. Pain can also be “caused” by the stimulation of nociceptive receptors and transmitted over intact neural pathways, such pain is termed “nociceptive” pain.

The level of stimulation at which pain becomes noted is referred to as the “pain threshold.” Analgesics are pharmaceutical agents which relieve pain by raising the pain threshold without a loss of consciousness. After administration of an analgesic drug, a stimulus of greater intensity or longer duration is required before pain is experienced. In an individual suffering from hyperalgesia an analgesic drug may have an anti-hyperalgesic effect. In contrast to analgesics, agents such as local anaesthetics block transmission in peripheral nerve fibers thereby blocking awareness of pain. General anaesthetics, on the other hand, reduce the awareness of pain by producing a loss of consciousness.

“Acute pain” is often short-lived with a specific cause and purpose; generally produces no persistent psychological reactions. Acute pain can occur during soft tissue injury, and with infection and inflammation. It can be modulated and removed by treating its cause and through combined strategies using analgesics to treat the pain and antibiotics to treat the infection.

“Chronic pain” is distinctly different from and more complex than acute pain. Chronic pain has no time limit, often has no apparent cause and serves no apparent biological purpose. Chronic pain can trigger multiple psychological problems that confound both patient and health care provider, leading to feelings of helplessness and hopelessness. The most common causes of chronic pain include low-back pain, headache, recurrent facial pain, pain associated with cancer and arthritis pain.

In one embodiment, the methods of the invention are used to treat “neuropathic pain.” Neuropathic pain typically is long-lasting or chronic and can develop days or months following an initial acute tissue injury. Symptoms of neuropathic pain can involve persistent, spontaneous pain, as well as allodynia, which is a painful response to a stimulus that normally is not painful, hyperalgesia, an accentuated response to a painful stimulus that usually a mild discomfort, such as a pin prick, or hyperpathia, a short discomfort becomes a prolonged severe pain. Neuropathic pain generally is resistant to opioid therapy. Neuropathic pain can be distinguished from nociceptive pain or “normal pain,” which is pain caused by the normal processing of stimuli resulting from acute tissue injury. In contrast to neuropathic pain, nociceptive pain usually is limited in duration to the period of tissue repair and usually can be alleviated by available opioid and non-opioid analgesics.

By “treating, reducing, or preventing pain” is meant preventing, reducing, or eliminating the sensation of pain in a subject before, during, or after it has occurred. As compared with an equivalent untreated control, such reduction or degree of prevention is at least 5%, 10%, 20%, 40%, 50%, 60%, 80%, 90%, 95%, or 100% as measured by any standard technique known in the art. To treat pain, according to the methods of this invention, the treatment does not necessarily provide therapy for the underlying pathology that is causing the painful sensation. Treatment of pain can be purely symptomatic.

In another embodiment of the claimed invention, the cooperativity between TRPA1 and TRPM5 can be used to amplify TRPM5 activation. Since TRPM5 is activated by intracellular calcium levels, an activator of TRPA1, which stimulates calcium influx, can be used to amplify TRPM5 activation. This TRPM5 amplification is useful for modulation of taste responses.

While specific configurations and methods describing the present invention are discussed, it should be understood that this is done for illustrative purposes only. A person skilled in the pertinent art will recognize that other configurations and methods can be used without departing from the spirit and scope of the present invention. It will be apparent to a person skilled in the pertinent art that this invention can also be employed in a variety of other applications.

### Cells

Cells for use in the method of the invention contain functional ion channels. The ion channels of the invention are TRPA1 and TRPM5 (“the ion channels”). The practitioner may use cells in which the ion channels are endogenous or may introduce either/both of the ion channels into a cell. If ion channels are endogenous to the cell, but the level of expression is not optimum, the practitioner may increase the level of expression of the ion channels in the cell. Where a given cell does not produce the ion channels at all, or at sufficient levels, a nucleic acid encoding the ion channels may be introduced into a host cell for expression and insertion into the cell membrane. The introduction, which may be generally referred to without limitation as “transformation,” may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. General aspects of mammalian cell host system transformations have been described in U.S. Pat. No. 4,399,216. For various techniques for transforming mammalian cells, see Keown et al., Meth. Enzym., 185: 527-537 (1990) and Mansour et al., Nature 336: 348-352 (1988).

TRPA1 (also known as p120, ANKTM1, CG5751, dTRPA1 and dANKTM1) is expressed as a 4.2 kb transcript in human tissues (Jaquemar, D., et al., J. Biol. Chem. 274: 7325-7333 (1999)). The open reading frame of the mRNA encodes a protein of 1119 amino acids forming two distinct domains. The amino-terminal domain consists of 18 repeats that are related to the cytoskeletal protein ankyrin. The carboxy-terminal domain contains six putative transmembrane segments that resemble many ion channels. The NCBI database lists several sequences for both the nucleic acid (10601, AE003554, AY496961, AK045771 and AY231177) and amino acid (CAA71610, AAF50356, AAS78661, BAC32487 and AA043183) sequences for many forms of TRPA1. The inclusion of the above sequences is for the purpose of illustration of the TRPA1 genetic sequence, however the invention is not to be limited to any one of the disclosed sequences.

TRPM5 (also known as TRP8, LTRPC5, MTR1 and 9430099A1Rik) is expressed as a 4.5 kb transcript in a variety of fetal and adult tissues (Prawitt et al. Hum. Mol. Gen. 9: 203-216 (2000)). Human TRPM5 has a putative reading frame containing 24 exons which encode an 1165 amino acid, membrane spanning polypeptide. The National Center for Biotechnology Information (NCBI) database lists several sequences for both the nucleic acid (NP_{—}064673, NP_{—}055370, AAP44477, AAP44476) and amino acid (NM_{—}014555, NM_{—}020277, AY280364, AY280365) sequences for both the human and mouse forms of TRPM5, respectively. The inclusion of the above sequences is for the purpose of illustration of the TRPM5 genetic sequence, however the invention is not to be limited to any one of the disclosed sequences.

It is recognized in the art that there can be significant heterogeneity in a gene sequence depending on the source of the isolated sequence. The invention contemplates the use of conservatively modified variants of the ion channels. Conservatively modified variants applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence to essentially identical sequences. Because of the degeneracy of the genetic code, a large number of functionally identical nucleic acids encode any given protein.

For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid, which encodes a polypeptide, is implicit in each described sequence.

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (I), Leucine (L), Methionine (M), Valine (V); and 6) Phenylalanine (F), Tyrosine (Y), Tryptophan (W); (see also, e.g., Creighton, Proteins, W. H. Freeman and Company (1984); Schultz and Schimer, Principles of Protein Structure, Springer-Verlag (1979)). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The variant ion channel proteins of the invention comprise non-conservative modifications (e.g. substitutions). By “nonconservative” modification herein is meant a modification in which the wildtype residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are nonconservative modifications. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine. In one embodiment, the variant ion channel proteins of the present invention have at least one nonconservative modification.

The variant proteins may be generated, for example, by using a PDA™ system previously described in U.S. Pat. Nos. 6,188,965; 6,296,312; 6,403,312; alanine scanning (see U.S. Pat. No. 5,506,107), gene shuffling (WO 01/25277), site saturation mutagenesis, mean field, sequence homology, polymerase chain reaction (PCR) or other methods known to those of skill in the art that guide the selection of point or deletion mutation sites and types.

The cells used in methods of the present invention may be present in, or extracted from, organisms, may be cells or cell lines transiently or permanently transfected or transformed with the appropriate ion channels or nucleic acids encoding them, or may be cells or cell lines that express the required ion channels from endogenous (i.e. not artificially introduced) genes.

Regulation of gene expression

Expression of the ion channel proteins refers to the translation of the ion channel polypeptides from an ion channel gene sequence either from an endogenous gene or from nucleic acid molecules introduced into a cell. The term “in situ” where used herein includes all these possibilities. Thus in situ methods may be performed in a suitably responsive cell line which expresses the ion channels. The cell line may be in tissue culture or may be, for example, a cell line xenograft in a non-human animal subject.

As used herein, the term “cell membrane” refers to a lipid bilayer surrounding a biological compartment, and encompasses an entire cell comprising such a membrane, or a portion of a cell.

For stable transfection of mammalian cells, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cell along with the gene of interest. Preferred selectable markers include those which confer resistance to drugs, such as G418, hygromycin and methotrexate. A nucleic acid encoding a selectable marker can be introduced into a host cell in the same vector as that encoding the ion channel proteins, or can be introduced in a separate vector. Cells stably transfected with the introduced nucleic acid can be identified by drug selection (e.g., cells that have incorporated the selectable marker gene will survive, while the other cells die).

It should be noted that expression of the ion channel proteins can also be controlled by any of a number of inducible promoters known in the art, such as a tetracycline responsive element, TRE. For example, the ion channel proteins can be selectively presented on the cell membrane by controlled expression using the Tet-on and Tet-off expression systems provided by Clontech (Gossen, M. and Bujard, H. Proc. Natl. Acad. Sci. USA 89: 5547-5551 (1992)). In the Tet-on system, gene expression is activated by the addition of a tetracycline derivative doxycycline (Dox), whereas in the Tet-off system, gene expression is turned on by the withdrawal of tetracyline (Tc) or Dox. Any other inducible mammalian gene expression system may also be used. Examples include systems using heat shock factors, steroid hormones, heavy metal ions, phorbol ester and interferons to conditionally expressing genes in mammalian cells.

The cell lines used in assays of the invention may be used to achieve transient expression of the ion channel proteins, or may be stably transfected with constructs that express an ion channel protein. Means to generate stably transformed cell lines are well known in the art, as well as described in U.S. Prov. Appl. No. 60/732,636, the disclosure of which is herein incorporated by reference, and such means may be used here. Examples of cells include, but are not limited to Chinese Hamster Ovary (CHO) cells, COS-7, HeLa, HEK 293, PC-12, and BAF.

The level of ion channel expression in a cell may be increased by introducing an ion channel nucleic acid into the cells or by causing or allowing expression from a heterologous nucleic acid encoding an ion channel. A cell may be used that endogenously expresses an ion channel without the introduction of heterologous genes. Such a cell may endogenously express sufficient levels of an ion channel for use in the methods of the invention, or may express only low levels of an ion channel which require supplementation as described herein.

The level of ion channel expression in a cell may also be increased by increasing the levels of expression of the endogenous gene. Endogenous gene activation techniques are known in the art and include, but are not limited to, the use of viral promoters (WO 93/09222; WO 94/12650 and WO 95/31560) and artificial transcription factors (Park et al. Nat. Biotech. 21: 1208-1214 (2003).

The level of ion channel expression in a cell may be determined by techniques known in the art, including but not limited to, nucleic acid hybridization, polymerase chain reaction, RNase protection, dot blotting, immunocytochemistry and Western blotting. Alternatively, ion channel expression can be measured using a reporter gene system. Such systems, which include for example red or green fluorescent protein (see, e.g. Mistili and Spector, Nature Biotechnology 15: 961-964 (1997), allow visualization of the reporter gene using standard techniques known to those of skill in the art, for example, fluorescence microscopy. Furthermore, the ability of TRPM5 to be activated by known positive modulating compounds, such as thrombin, may be determined following manipulation of the ion channel expressing cells.

Cells described herein may be cultured in any conventional nutrient media. The culture conditions, such as media, temperature, pH and the like, can be selected by the skilled artisan without undue experimentation. In general, principles, protocols, and practical techniques for maximizing the productivity of cell cultures can be found in “Mammalian Cell Biotechnology: a Practical Approach”, M. Butler, ed. JRL Press, (1991) and Sambrook et al, supra.

The cells can be grown in solution or on a solid support. The cells can be adherent or non-adherent. Solid supports include glass or plastic culture dishes, and plates having one compartment, or multiple compartments, e.g., multi-well plates. The multi-well vessels of the claimed invention may contain up to and a number equaling 96 wells. In another embodiment, the multi-well vessel comprises greater than 96 wells. In another embodiment, the multi-well vessel comprises 384 wells. In yet another embodiment, the multi-well vessel comprises 1536 wells.

The number of cells seeded into each well are preferably chosen so that the cells are at or near confluence, but not overgrown, when the assays are conducted, so that the signal-to-background ratio of the signal is increased.

In one embodiment of the present invention, inhibitors of gene expression of one ion channel are used to reduce gene expression of the other channel. “Reduce gene expression” as used herein refers to reduction in the level of MRNA, protein, or both MRNA and protein, encoded by a gene or nucleotide sequence of interest. Reduction of gene expression may arise as a result of the lack of production of full length RNA.

In one embodiment, an inhibitor is a nucleic acid, for example, an anti-sense nucleotide sequence, an RNA molecule, or an aptamer sequence. An anti-sense nucleotide sequence can bind to a nucleotide sequence within a cell and modulate the level of expression of a persistent sodium channel gene, or modulate expression of another gene that controls the expression or activity of a persistent sodium channel. Similarly, an RNA molecule, such as a catalytic ribozyme, can bind to and alter the expression of a persistent sodium channel gene, or other gene that controls the expression or activity of a persistent sodium channel. An aptamer is a nucleic acid sequence that has a three dimensional structure capable of binding to a molecular target, see, e.g., Jayasena, S. D. Clin. Chem. 45: 1628-1650 (1999).

In addition, a selective antagonist can also be a double-stranded RNA molecule for use in RNA interference methods. RNA interference (RNAI) is a process of sequence-specific gene silencing by post-transcriptional RNA degradation, which is initiated by double-stranded RNA (dsRNA) homologous in sequence to the silenced gene. A suitable double-stranded RNA (dsRNA) for RNAI contains sense and antisense strands of, for example, about 21 contiguous nucleotides corresponding to the gene to be targeted that form 19 RNA base pairs, leaving overhangs of two nucleotides at each 3′ end (Elbashir, S. M. et al., Nature 411: 494-498 (2001); Bass, B. L. Nature 411: 428-429 (2001); Zamore, P. D. Nat. Struct. Biol. 8: 746-750 (2001). dsRNAs of about 25-30 nucleotides have also been used successfully for RNAi (Karabinos, A. et al., Proc. Natl. Acad. Sci. USA 98: 7863-7868 (2001). dsRNA can be synthesized in vitro and introduced into a cell by methods known in the art.

Antibodies can also be used as an antagonist of ion channel expression. As used herein, the term “antibody” is meant to include polyclonal antibodies, monoclonal antibodies (mAbs), chimeric antibodies, anti-idiotypic (anti-Id) antibodies to antibodies that can be labeled in soluble or bound form, as well as fragments thereof provided by any known technique, such as, but not limited to enzymatic cleavage, peptide synthesis or recombinant techniques.

Polyclonal antibodies are heterogeneous populations of antibody molecules derived from the sera of animals immunized with an antigen. A monoclonal antibody (mAb) contains a substantially homogeneous population of antibodies specific to antigens, which populations contains substantially similar epitope binding sites. MAbs may be obtained by methods known to those skilled in the art. See, for example Kohler, G. et al., Nature 256: 495-497 (1975); U.S. Pat. No. 4,376,110. Such antibodies may be of any immunoglobulin class including IgG, IgM, IgE, IgA, GILD and any subclass thereof. A hybridoma producing a mAb of the present invention may be cultivated in vitro, in situ or in vivo. Production of high titers of mAbs in vivo or in situ makes this the presently preferred method of production.

### Ion Channel Activation

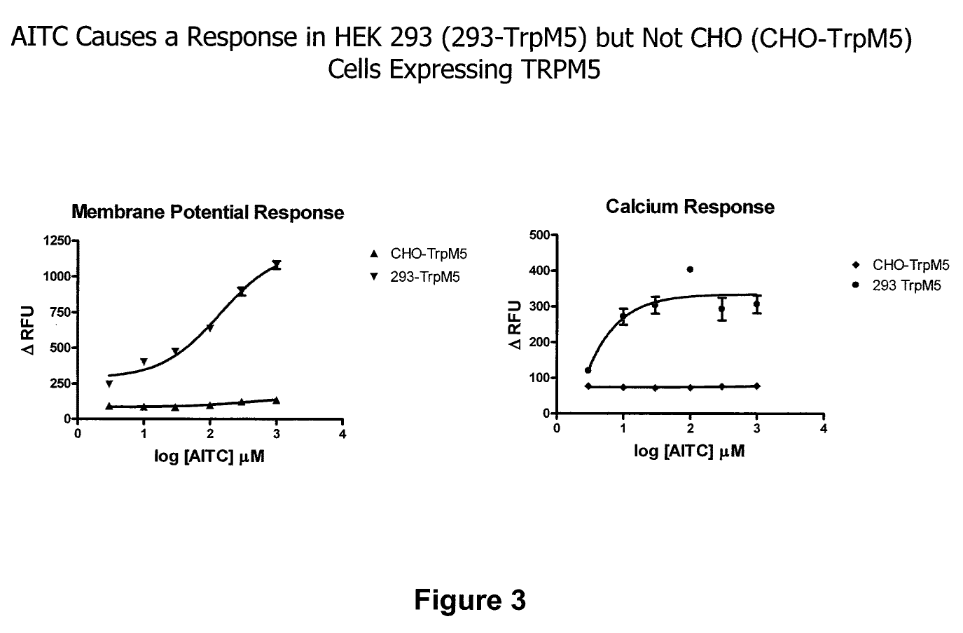

In order to observe ion channel activity, and evaluate whether a test compound can modulate activation, cells expressing the ion channels must be exposed to an activator. For the TRPM5 ion channel, intracellular calcium activators are used. As mentioned above, TRPA1 is activated by several types of compounds including natural compounds, cold temperatures and environmental irritants. Natural compounds include, but are not limited to cinnamaldehyde, eugenol, gingerol, methyl salicylate, AITC and allicin. There are many methods to activate intracellular calcium stores and many calcium activating agents are known in the art and include, but are not limited to thrombin, adenosine triphosphate (ATP), carbachol, and calcium ionophores (e.g. A23187). While nanomolar increases in calcium concentration ranges are required for TRPM5 channel activation, the concentration ranges useful for the claimed invention are known in the art, e.g., between 10⁻¹⁰ to 10⁻⁴ M for ATP. However, the precise concentration may vary depending on a variety of factors including cell type and time of incubation. The increased calcium concentration can be confirmed using calcium sensitive dyes, e.g., Fluo 3, Fluo 4, or FLIPR calcium 3 dye and single cell imaging techniques in conjunction with Fura2. Changes in membrane potential can also be controlled using cells that cannot generate a TRPM5 response such as TRPM5-CHO cells (see FIG. 3).

Test cells can also be incubated with lower doses of the calcium activating agents described above, such that a fluorescent response that is lower than the maximum achievable response is generated. Generally, the dose is referred to as the effect concentration or EC₂₀₋₃₀, which relates to the effect condition where the fluorescent intensity is 20-30% of the maximal response. As used herein, “EC” refers to effect condition, such that EC₂₀ refers to the effect condition where the fluorescent intensity is 20% of the maximal response is generated. Upon the addition of a second ion channel-specific activating compound, this low response will be increased to at, or near, maximal levels of activation.

In general, agonists and antagonists are used to modulate the ion channels. Agonists” are molecules or compounds that stimulate one or more of the biological properties of a polypeptide of the present invention. These may include, but are not limited to, small organic and inorganic molecules, peptides, peptide mimetics and agonist antibodies. The term “antagonist” is used in the broadest sense and refers to any molecule or compound that blocks, inhibits or neutralizes, either partially or fully, a biological activity mediated by a receptor of the present invention by preventing the binding of an agonist. Antagonists may include, but are not limited to, small organic and inorganic molecules, peptides, peptide mimetics and neutralizing antibodies.

### Detection of Ion Channel Activation

Movement of physiologically relevant substrates through ion channels can be traced by a variety of physical, optical, or chemical techniques (Stein, W. D., Transport and Diffusion Across Cell Membranes, 1986, Academic Press, Orlando, Fla.). Assays for modulators of ion channels include electrophysiological assays, cell-by-cell assays using microelectrodes (Wu, C. -F. et al., Neurosci 3(9): 1888-99 (1983)), i.e., intracellular and patch clamp techniques (Neher, E. and Sakmann, B., Sci. Amer. 266: 44-51 (1992)), and radioactive tracer ion techniques. Preferably, the effect of the candidate compound is determined by measuring the change in the cell membrane potential after the cell is exposed to the compound. This may be done, for example, using a fluorescent dye that emits fluorescence in response to changes in cell membrane potential and an optical reader to detect this fluorescence.

Optical methods using fluorescence detection are particularly suitable methods for high throughput screening of candidate compounds. Optical methods permit measurement of the entire course of ion flux in a single cell as well as in groups of cells. The advantages of monitoring transport by fluorescence techniques include the high level of sensitivity of these methods, temporal resolution, modest demand for biological material, lack of radioactivity, and the ability to continuously monitor ion transport to obtain kinetic information (Eidelman, O. et al., Biophys. Acta 988: 319-334 (1989)). Present day optical readers detect fluorescence from multiple samples in a short time and can be automated. Fluorescence readouts are used widely both to monitor intracellular ion concentrations and to measure membrane potentials.

Voltage sensitive dyes that may be used in the assays and methods of the invention have been used to address cellular membrane potentials (Zochowski et al., Biol. Bull. 198: 1-21 (2000)). Membrane potential dyes or voltage-sensitive dyes refer to molecules or combinations of molecules that enter depolarized cells, bind to intracellular proteins or membranes and exhibit enhanced fluorescence. These dyes can be used to detect changes in the activity of an ion channel such as TRPM5, expressed in a cell. Voltage-sensitive dyes include, but are not limited to, modified bisoxonol dyes, sodium dyes, potassium dyes and thorium dyes. The dyes enter cells and bind to intracellular proteins or membranes, therein exhibiting enhanced fluorescence and red spectral shifts (Epps et al., Chem. Phys. Lipids 69: 137-150 (1994)). Increased depolarization results in more influx of the anionic dye and thus an increase in fluorescence.

In one embodiment, the membrane potential dyes are FMP dyes available from Molecular Devices (Catalog Nos. R8034, R8123). In other embodiments, suitable dyes could include dual wavelength FRET-based dyes such as DiSBAC2, DiSBAC3, and CC-2-DMPE (Invitrogen Cat. No. K1016). [Chemical Name Pacific Blue™ 1,2-ditetradecanoyl-sn-glycero-3-phosphoethanolamine, triethylammonium salt].

Calcium-sensitive fluorescent agents are also useful to detect changes in TRPA1 activity. Suitable types of calcium-sensitive fluorescent agents include Fluo3, Fluo4, Fluo5, Calcium Green, Calcium Orange, Calcium Yellow, Fura-2, Fura-4, Fura-5, Fura-6, Fura-FF, Fura Red, indo-1, indo-5, BTC (Molecular Probes, Eugene, Oreg.), and FLIPR Calcium3 wash-free dye (Molecular Devices, Sunnyvale Calif.). In one embodiment, the intracellular calcium dye is the FLIPR Calcium 3 dye available from Molecular Devices (Part Number: R8091). Additional calcium-sensitive fluorescent agents known to the skilled artisan are also suitable for use in the claimed assay. The calcium-sensitive fluorescent agents can be hydrophilic or hydrophobic.

Sodium-sensitive fluorescent agents are also useful to detect changes in TRPA1 activity. Suitable types of sodium-sensitive fluorescent agents include CoroNa™ Green, CoroNa™ Red chloride, SBFI, and Sodium Green™ (Molecular Probes, Eugene, Oreg.). Additional sodium-sensitive fluorescent agents known to the skilled artisan are also suitable for use in the claimed assay. The sodium-sensitive fluorescent agents can be hydrophilic or hydrophobic.

The voltage- or ion-sensitive fluorescent dyes are loaded into the cytoplasm by contacting the cells with a solution comprising a membrane-permeable derivative of the dye. However, the loading process may be facilitated where a more hydrophobic form of the dye is used. Thus, voltage- and ion-sensitive fluorescent dyes are known and available as hydrophobic acetoxymethyl esters, which are able to permeate cell membranes more readily than the unmodified dyes. As the acetoxymethyl ester form of the dye enters the cell, the ester group is removed by cytosolic esterases, thereby trapping the dye in the cytosol.

The ion channel-expressing cells of the assay are generally preloaded with the fluorescent dyes for 30-240 minutes prior to addition of candidate compounds. Preloading refers to the addition of the fluorescent dye for a period prior to candidate compound addition during which the dye enters the cell and binds to intracellular lipophilic moieties. Cells are typically treated with 1 to 10 μM buffered solutions of the dye for 20 to 60 minutes at 37° C. In some cases it is necessary to remove the dye solutions from the cells and add fresh assay buffer before proceeding with the assay.

Another method for testing ion channel activity is to measure changes in cell membrane potential using the patch-clamp technique. (Hamill et al., Nature 294: 462-4 (1981)). In this technique, a cell is attached to an electrode containing a micropipette tip which directly measures the electrical conditions of the cell. This allows detailed biophysical characterization of changes in membrane potential in response to various stimuli. Thus, the patch-clamp technique can be used as a screening tool to identify compounds that modulate activity of ion channels.

Radiotracer ions have been used for biochemical and pharmacological investigations of channel-controlled ion translocation in cell preparations (Hosford, D. A. et al., Brain Res. 516: 192-200 (1990)). In this method, the cells are exposed to a radioactive tracer ion and an activating ligand for a period of time, the cells are then washed, and counted for radioactive content. Radioactive isotopes are well known (Evans, E. A., Muramtsu, M. Radiotracer Techniques and Applications, M. Dekker, New York (1977)) and their uses have permitted detection of target substances with high sensitivity. As used herein, the phrase “screening for inhibitors of TRPA1 activity” refers to use of an appropriate assay system to identify novel TRPA1 modulators from test agents. The assay can be an in vitro or an in vivo assay suitable for identifying whether a test agent can stimulate or suppress one or more of the biological functions of a TRPA1 molecule or a phospholipase C (PLC) polypeptide. Examples of suitable bioassays include, but are not limited to, assays for examining binding of test agents to a PLC polypeptide or a TRPA1 polypeptide (e.g., a TRPA1 fragment containing its ligand binding domain), calcium influx assay, or behavioral analysis. Either an intact PLC or TRPA1 polypeptide or polynucleotide, fragments, variants, or substantially identical sequences may be used in the screening.

### Assay Detection

Detecting and recording alterations in the spectral characteristics of the dye in response to changes in membrane potential may be performed by any means known to those skilled in the art. As used herein, a “recording” refers to collecting and/or storing data obtained from processed fluorescent signals, such as are obtained in fluorescent imaging analysis.

In some embodiments, the assays of the present invention are performed on isolated cells using microscopic imaging to detect changes in spectral (i.e., fluorescent) properties. In other embodiments, the assay is performed in a multi-well format and spectral characteristics are determined using a microplate reader.

By “well” it is meant generally a bounded area within a container, which may be either discrete (e.g., to provide for an isolated sample) or in communication with one or more other bounded areas (e.g., to provide for fluid communication between one or more samples in a well). For example, cells grown on a substrate are normally contained within a well that may also contain culture medium for living cells. Substrates can comprise any suitable material, such as plastic, glass, and the like. Plastic is conventionally used for maintenance and/or growth of cells in vitro.

A “multi-well vessel”, as noted above, is an example of a substrate comprising more than one well in an array. Multi-well vessels useful in the invention can be of any of a variety of standard formats (e.g., plates having 2, 4, 6, 24, 96, 384, or 1536, etc., wells), but can also be in a non-standard format (e.g., plates having 3, 5, 7, etc., wells).

A suitable configuration for single cell imaging involves the use of a microscope equipped with a computer system. One example of such a configuration, ATTO's Attofluor® RatioVision® real-time digital fluorescence analyzer from Carl Zeiss, is a completely integrated work station for the analysis of fluorescent probes in living cells and prepared specimens (ATTO, Rockville, Md.). The system can observe ions either individually or simultaneously in combinations limited only by the optical properties of the probes in use. The standard imaging system is capable of performing multiple dye experiments such as FMP (for sodium) combined with GFP (for transfection) in the same cells over the same period of time. Ratio images and graphical data from multiple dyes are displayed online.

When the assays of the invention are performed in a multi-well format, a suitable device for detecting changes in spectral qualities of the dyes used is a multi-well microplate reader. Suitable devices are commercially available, for example, from Molecular Devices (FLEXstation® microplate reader and fluid transfer system or FLIPR® system), from Hamamatsu (FDSS 6000) and the “VIPR” voltage ion probe reader (Aurora, Bioscience Corp. Calif., USA). The FLIPR-Tetra™ is a second generation reader that provides real-time kinetic cell-based assays using up to 1536 simultaneous liquid transfer systems. All of these systems can be used with commercially available dyes such as FMP, which excites in the visible wavelength range.

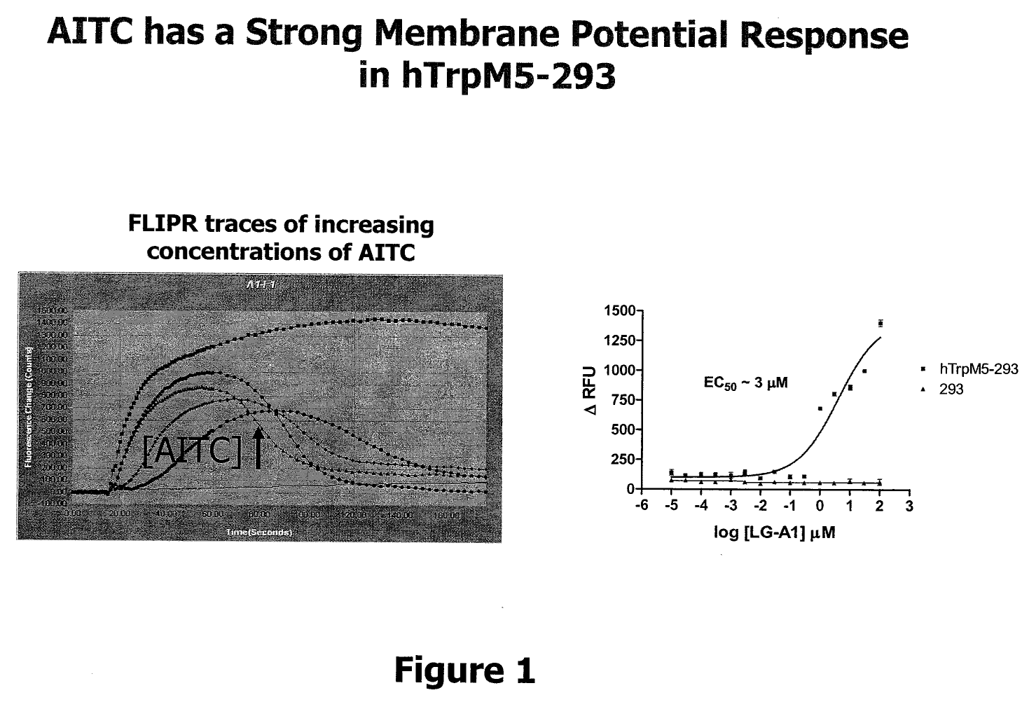

Using the FLIPR® system, the change in fluorescent intensity is monitored over time and is graphically displayed as shown, for example in FIG. 1. The addition of ion channel enhancing compounds causes an increase in fluorescence, while ion channel blocking compounds block this increase.

Several commercial fluorescence detectors are available that can inject liquid into a single well or simultaneously into multiple wells. These include, but are not limited to, the Molecular Devices FlexStation (eight wells), BMG NovoStar (two wells) and Aurora VIPR (eight wells). Typically, these instruments require 12 to 96 minutes to read a 96-well plate in flash luminescence or fluorescence mode (1 min/well). An alternative method is to inject the modulator into all sample wells at the same time and measure the luminescence in the whole plate by imaging with a charge-coupled device (CCD) camera, similar to the way that calcium responses are read by calcium-sensitive fluorescent dyes in the FLIPR®, FLIPR-384 or FLIPR-Tetra™ instruments. Other fluorescence imaging systems with integrated liquid handling are expected from other commercial suppliers such as the second generation LEADSEEKER from Amersham, the Perkin Elmer CellLux—Cellular Fluorescence Workstation and the Hamamatsu FDSS6000 System. These instruments can generally be configured to proper excitation and emission settings to read FMP dye (540ₑₓ±15 nm, 570ₑₘ±15 nm) and calcium dye (490ₑₓ±15 nm, 530ₑₘ±15 nm). The excitation/emission characteristics differ for each dye, therefore, the instruments are configured to detect the dye chosen for each assay.

The data generated by the optical detectors can be processed using a variety of computerized programs known in the art. For example, time-sequence files generated by the FLIPR® system can be processed using the data reduction package CeuticalSoft®. The CeuticalSoft® data package consists of: Kinetiture®, which views the kinetic traces, extracts FLIPR peak heights and marks outliers; Calcature®, which calculates normalized response (percent of control) for agonist assay (1st addition) and antagonist assay (2nd addition); and Curvature®, which calculates effective concentration for 50% activation (EC₅₀) and concentration for 50% inhibition (IC₅₀). The processed data can be stored in searchable databases, such as the Microsoft Access Database.

Finally, cheminformatics analysis can be performed using a 2D/3D cluster analysis of active structures within and between taste receptor (TRP) assays to group similar molecules. Models of compound structure versus comparative TRP channel activation can be created to assist in the potential identification of new TRP channel activating molecules.

### Candidate Compounds

Candidate compounds employed in the screening methods of this invention include for example, without limitation, synthetic organic compounds, chemical compounds, naturally occurring products, polypeptides and peptides, nucleic acids, etc.

Essentially any chemical compound can be used as a potential modulator or ligand in the assays of the invention. Most often compounds dissolved in aqueous or organic (especially dimethyl sulfoxide- or DMSO-based) solutions are used. The assays are designed to screen large chemical libraries by automating the assay steps. The compounds are provided from any convenient source to the cells. The assays are typically run in parallel (e.g., in microtiter formats on microtiter plates in robotic assays with different test compounds in different wells on the same plate). It will be appreciated that there are many suppliers of chemical compounds, including ChemDiv (San Diego, Calif.), Sigrna-Aldrich (St. Louis, Mo.), Fluka Chemika-Biochemica-Analytika (Buchs Switzerland) and the like.

“Modulating” as used herein includes any effect on the functional activity of the ion channels. This includes blocking or inhibiting the activity of the channel in the presence of, or in response to, an appropriate stimulator. Alternatively, modulators may enhance the activity of the channel. “Enhance” as used herein, includes any increase in the functional activity of the ion channels.

In one embodiment, the high throughput screening methods involve providing a small organic molecule or peptide library containing a large number of potential ion channel modulators. Such “chemical libraries” are then screened in one or more assays, as described herein, to identify those library members (particular chemical species or subclasses) that display a desired characteristic activity. The compounds thus identified can serve as conventional “lead compounds” or can themselves be used as potential or actual products.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka Int. J. Pept. Prot. Res. 37: 487-493 (1991) and Houghton et al., Nature 354: 84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913 (1993)), vinylogous polypeptides (Hagihara et al., J. Amer. Chem. Soc. 114: 6568 (1992)), nonpeptidal peptidomimetics with glucose scaffolding (Hirschmann et al., J. Amer. Chem. Soc. 114: 9217-9218 (1992)), analogous organic syntheses of small compound libraries (Chen et al., J. Amer. Chem. Soc. 116: 2661 (1994)), oligocarbamates (Cho et al., Science 261: 1303 (1993)), and/or peptidyl phosphonates (Campbell et al., J. Org. Chem. 59: 658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14: 309-314 (1996) and PCT/US96/10287), carbohydrate libraries (see, e.g., Liang et al., Science, 274: 1520-1522 (1996) and U.S. Pat. No. 5,593,853), small organic molecule libraries (see, e.g., isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Asinex, Moscow, Russia; Tripos, Inc., St. Louis, Mo.; ChemStar, Ltd, Moscow, Russia; 3D Pharmaceuticals, Exton, Pa.; Martek Biosciences, Columbia, Md.; etc.).

Candidate agents, compounds, drugs, and the like encompass numerous chemical classes, though typically they are organic molecules, preferably small organic compounds having a molecular weight of more than 100 and less than about 10,000 daltons, preferably, less than about 2000 to 5000 daltons. Candidate compounds may comprise functional groups necessary for structural interaction with proteins, particularly hydrogen bonding, and typically include at least an amine, carbonyl, hydroxyl or carboxyl group, preferably at least two of the functional chemical groups. The candidate compounds may comprise cyclical carbon or heterocyclic structures, and/or aromatic or polyaromatic structures substituted with one or more of the above functional groups. Candidate compounds are also found among biomolecules including peptides, saccharides, fatty acids, steroids, purines, pyrimidines, derivatives, structural analogs or combinations thereof.

A variety of other reagents may be included in the screening assay according to the present invention. Such reagents include, but are not limited to, salts, solvents, neutral proteins, e.g. albumin, detergents, etc., which may be used to facilitate optimal protein-protein binding and/or to reduce non-specific or background interactions. Examples of solvents include, but are not limited to, dimethyl sulfoxide (DMSO), ethanol and acetone, and are generally used at a concentration of less than or equal to 1% (v/v) of the total assay volume. In addition, reagents that otherwise improve the efficiency of the assay, such as protease inhibitors, anti-microbial agents, etc. may be used. Further, the mixture of components in the method may be added in any order that provides for the requisite binding.

The compounds identified using the disclosed assay are potentially useful as ingredients or flavorants in ingestible compositions, i.e., foods and beverages as wells as orally administered medicinals. Compounds that modulate taste perception can be used alone or in combination as flavorants in foods or beverages. The amount of such compound(s) will be an amount that yields the desired degree of modulated taste perception of which starting concentrations may generally be between 0.1 and 1000 μM.

### Pain Models

The ability of a compound that selectively reduces TRPA1-mediated pain can be confirmed using a variety of well-known assays.

Tail Flick Model: The tail-flick test (D'Amour et al., J. Pharmacol. Exp. and Ther. 72: 74-79 (1941)) is a model of acute pain. A gently-restrained rat is placed on a test stage such that a focused light source beams on the dorsal or ventral surface of the rat's tail. A photosensor is present on the test stage located opposite the light source. To begin the test, the rat's tail blocks the light, thus preventing the light reaching the photosensor. Latency measurement begins with the activation of the light source. When a rat moves or flicks its tail, the photosensor detects the light source and stops the measurement. The test measures the period of time (duration) that the rat's tail remains immobile (latent). Rats are tested prior to administration thereto of a compound of interest and then at various times after such administration.

Rat Tail Immersion Model: The rat tail immersion assay is also a model of acute pain. A rat is loosely held in hand while covered with a small folded thin cotton towel with its tail exposed. The tip of the tail is dipped into a, e.g., 52° C. water bath to a depth of two inches. The rat responds by either wiggling of the tail or withdrawal of the tail from the water; either response is scored as the behavioral end-point. Rats are tested for a tail response latency (TRL) score prior to administration thereto of a compound of interest and then retested for TRL at various times after such administration.

Carrageenan-induced Paw Hyperalgesia Model: The carrageenan paw hyperalgesia test is a model of inflammatory pain. A subcutaneous injection of carrageenan is made into the left hindpaws of rats. The rats are treated with a selected agent before, e.g., 30 minutes, the carrageenan injection or after, e.g., two hours after, the carrageenan injection. Paw pressure sensitivity for each animal is tested with an analgesymeter three hours after the carrageenan injection. See, Randall et al., Arch. Int. Pharmacodyn. 111: 409-419 (1957).

The effects of selected agents on carrageenan-induced paw edema can also be examined. This test (see, Vinegar et al., J. Phamacol. Exp. Ther. 166: 96-103 (1969) allows an assessment of the ability of a compound to reverse or prevent the formation of edema evoked by paw carrageenan injection. The paw edema test is carried out using a plethysmometer for paw measurements. After administration of a selected agent, a carrageenan solution is injected subcutaneously into the lateral foot pad on the plantar surface of the left hind paw. At three hours post-carrageenan treatment, the volume of the treated paw (left) and the untreated paw (right) is measured using a plethysmometer.

Formalin Behavioral Response Model: The formalin test is a model of acute, persistent pain. Response to formalin treatment is biphasic (Dubuisson et al., Pain 4: 161-174 (1977)). The Phase I response is indicative of a pure nociceptive response to the irritant. Phase 2, typically beginning 20 to 60 minutes following injection of formalin, is thought to reflect increased sensitization of the spinal cord.

Von Frey Filament Test (Chang model): The effect of compounds on mechanical allodynia can be determined by the von Frey filament test in rats with a tight ligation of the L-5 spinal nerve: a model of painful peripheral neuropathy. The surgical procedure is performed as described by Kim et al., Pain 50: 355-363 (1992). A calibrated series of von Frey filaments are used to assess mechanical allodynia (Chaplan et al., J. Neurosci. Methods 53: 55-63 (1994)). Filaments of increasing stiffness are applied perpendicular to the midplantar surface in the sciatic nerve distribution of the left hindpaw. The filaments are slowly depressed until bending occurred and are then held for 4-6 seconds. Flinching and licking of the paw and paw withdrawal on the ligated side are considered positive responses.

Chronic Constriction Injury: Heat and cold allodynia responses can be evaluated as described below in rats having a chronic constriction injury (CCI). A unilateral mononeuropathy is produced in rats using the chronic constriction injury model described in Bennett et al., Pain 33: 87-107 (1988). CCI is produced in anesthetized rats as follows. The lateral aspect of each rat's hind limb is shaved and scrubbed with Nolvasan. Using aseptic techniques, an incision is made on the lateral aspect of the hind limb at the mid-thigh level. The biceps femoris is bluntly dissected to expose the sciatic nerve. On the right hind limb of each rat, four loosely tied ligatures (for example, Chromic gut 4.0; Ethicon, Johnson and Johnson, Somerville, N.J.) are made around the sciatic nerve approximately 1-2 mm apart. On the left side of each rat, an identical dissection is performed except that the sciatic nerve is not ligated (sham). The muscle is closed with a continuous suture pattern with, e.g., 4-0 Vicryl (Johnson and Johnson, Somerville, N.J.) and the overlying skin is closed with wound clips. The rats are ear-tagged for identification purposes and returned to animal housing.

The Hargreaves Test: The Hargreaves test (Hargreaves et al., Pain 32: 77-88 (1998)) is also a radiant heat model for pain. CCI rats are tested for thermal hyperalgesia at least 10 days post-op. The test apparatus consists of an elevated heated (80-82° F.) glass platform. Eight rats at a time, representing all testing groups, are confined individually in inverted plastic cages on the glass floor of the platform at least 15 minutes before testing. A radiant heat source placed underneath the glass is aimed at the plantar hind paw of each rat. The application of heat is continued until the paw is withdrawn (withdrawal latency) or the time elapsed is 20 seconds. This trial is also applied to the sham operated leg. Two to four trials are conducted on each paw, alternately, with at least 5 minutes interval between trials. The average of these values represents the withdrawal latency.

Cold Allodynia Model: The test apparatus and methods of behavioral testing is described in Gogas et al., Analgesia 3: 111-118 (1997). The apparatus for testing cold allodynia in neuropathic (CCI) rats consists of a Plexiglass chamber with a metal plate 6 cm from the bottom of the chamber. The chamber is filled with ice and water to a depth of 2.5 cm above the metal plate, with the temperature of the bath maintained at 0-4° C. throughout the test. Each rat is placed into the chamber individually, a timer started, and the animal's response latency was measured to the nearest tenth of a second. A “response” is defined as a rapid withdrawal of the right ligated hindpaw completely out of the water when the animal is stationary and not pivoting. An exaggerated limp while the animal is walking and turning is not scored as a response. The animals' baseline scores for withdrawal of the ligated leg from the water typically range from 7-13 seconds. The maximum immersion time is 20 seconds with a 20-minute interval between trials.

Using any of these assays and others known in the art, those skilled in the art recognize that ED₅₀ values and their standard errors of the mean can be determined using accepted numerical methods, see, e.g., Roger E. Kirk, Experimental Design: Procedures for the Behavioral Sciences, (Wadsworth Publishing, 3^{rd} ed. 1994).

### Pharmaceutical Compositions

As disclosed herein, a selective modulator of TRPM5 can be administered to a mammal to modulate in vivo processes involving TRPA1 such as treating pain, mechanosensation and modifying taste. As used herein, the term “treating pain,” when used in reference to administering to a mammal an effective amount of a TRPM5 antagonist, means reducing a symptom of pain, or delaying or preventing onset of a symptom of pain in the mammal. The effectiveness of a TRPM5 antagonist in treating pain can be determined by observing one or more clinical symptoms or physiological indicators associated with pain, as described above.

The appropriate effective amount to be administered for a particular application of the methods can be determined by those skilled in the art, using the guidance provided herein. For example, an effective amount can be extrapolated from in vitro and in vivo assays as described herein above. One will recognize that the condition of the patient can be monitored throughout the course of therapy and that the effective amount of a TRPM5 antagonist that is administered can be adjusted accordingly.

The invention also can be practiced by administering an effective amount of a TRPM5 antagonist together with one or more other agents including, but not limited to, one or more analgesic agents. In such “combination” therapy, it is understood that the antagonist can be delivered independently or simultaneously, in the same or different pharmaceutical compositions, and by the same or different routes of administration as the one or more other agents.

A TRPM5 antagonist or other compound useful in the invention generally is administered in a pharmaceutical acceptable composition. As used herein, the term “pharmaceutically acceptable” refer to any molecular entity or composition that does not produce an adverse, allergic or other untoward or unwanted reaction when administered to a human or other mammal. As used herein, the term “pharmaceutically acceptable composition” refers to a therapeutically effective concentration of an active ingredient. A pharmaceutical composition may be administered to a patient alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

It is also envisioned that a pharmaceutical composition can optionally include a pharmaceutically acceptable carriers that facilitate processing of an active ingredient into pharmaceutically acceptable compositions. As used herein, the term “pharmacologically acceptable carrier” refers to any carrier that has substantially no long term or permanent detrimental effect when administered and encompasses terms such as “pharmacologically acceptable vehicle, stabilizer, diluent, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., distilled, deionized water, saline; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical dosage forms and drug delivery systems (Ansel, H. C. et al., eds., Lippincott Williams & Wilkins Publishers, 7^{th} ed. 1999); Remington: The Science and Practice of Pharmacy (Gennaro, A. R. ed., Lippincott, Williams & Wilkins, 20^{th} ed. 2000); Goodman & Gilman's The Pharmacological Basis of Therapeutics (Hardman, J. G. et al., eds., McGraw-Hill Professional, 10^{th} ed. 2001); and Handbook of Pharmaceutical Excipients (Rowe, R. C. et al., APhA Publications, 4^{th} edition 2003).

It is further envisioned that a pharmaceutical composition disclosed in the present specification can optionally include, without limitation, other pharmaceutically acceptable components, including, without limitation, buffers, preservatives, tonicity adjusters, salts, antioxidants, physiological substances, pharmacological substances, bulking agents, emulsifying agents, wetting agents, sweetening or flavoring agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy-chloro composition. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms.

An antagonist useful in a method of the invention is administered to a mammal in an effective amount. Such an effective amount generally is the minimum dose necessary to achieve the desired effect, which can be, for example for treating pain, that amount roughly necessary to reduce the discomfort caused by the pain to tolerable levels or to achieve a significant reduction in pain. For example, the term “effective amount” when used with respect to treating pain can be a dose sufficient to reduce pain, for example, by at least 30%, 40%, 50%, 60%, 70%, 80%, 90% or 100%. The subject receiving the TRPM5 antagonist can be any mammal or other vertebrate in which modulation of TRPA1-associated processes is desired, for example, a human, primate, horse, cow, dog, cat or bird.

Various routes of administration can be useful according to a method of the invention. Routes of peripheral administration useful in the methods of the invention encompass, without limitation, oral administration, topical administration, intravenous or other injection, and implanted minipumps or other extended release devices or formulations. A pharmaceutical composition useful in the invention can be peripherally administered, for example, orally in any acceptable form such as in a tablet, liquid, capsule, powder, or the like; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation.

### EXAMPLES

### Example 1

### The TRPA1 Activator AITC Causes Strong Membrane Potential and Calcium Responses in TRPM5-expressing Cells

As described in greater detail below, HEK 293 cells transfected with a plasmid bearing the human TRPM5 gene, were used to identify the cooperativity that exists between TRPA1 and TRPM5.

### Plasmid Construction

First strand cDNA was synthesized by Thermoscript RT-PCR System (Invitrogen) from human small intestine poly A+ RNA (BD Biosciences) and the full length hTRPM5 was amplified by PCR using GC Melt (BD Biosciences). The product was PCR purified by Pure Link PCR Purification (Invitrogen) and inserted into a vector using the TOPO TA Cloning Kit (Invitrogen). After sequencing, 6 mutations were found and the mutations were corrected using the Quick Change Multi Site Directed Mutagenesis Kit (Stratagene) in 2 rounds. Three mutations were corrected in each round. The fall length TRPM5 was excised from the TOPO TA vector using the EcoRI and NotI restriction enzymes and ligated in the pENTR 3C vector, which had also been digested with EcoRI and NotI. The insert and vector bands were gel extracted and purified using the SNAP Gel Purification Kit (Invitrogen). Finally, LR Recombination Reaction (Invitrogen) was used to insert the entry clone into destination vectors of interest (e.g., pT-Rex-DEST 30, pcDNA-DEST 53, pcDNA 3.2/v5-DEST and pcDNA 6.2/V5-DEST).

Development of hTRPM5 Stable Cell Line

To create a stably transfected cell line expressing hTRPM5, 1.0×10⁶ HEK 293 cells (ATCC, Manassas, Va.) were seeded in 35 mm tissue culture dishes (Falcon, BD Biosciences, Bedford, Mass.) and grown overnight in a 37° C. and 5% CO₂ incubator, in culture medium consisting of DMEM, 10% fetal bovine serum (FBS), and penicillin with streptomycin. The next day, the cells were transfected using 4 μg of pcDNA 3.2-hTRPM5 with 7 μl of Lipofectamine 2000 (Invitrogen), following the manufacturer's protocol. After two days in culture, the cells were replated at 1:10 and 1:100 dilution, and from those plates seeded at very low density in 96-well plates to isolate single-cell colonies. To select individual clones, 1 mg/ml Geneticin (Invitrogen) was added to the culture medium. Once stably expressing clones were identified, the concentration of Geneticin was reduced to 0.25 mg/ml for expansion and maintenance. Clones were selected on the basis of their response to ATP (Sigma) and lonomycin (Sigma) in the FLIPR® assay using the Membrane Potential Assay Kit RED (Molecular Devices). Stable cell lines in chinese hamster ovary cells (CHO-M1) were created similarly except that 5.0×10⁵ cells were plated in 35 mm dishes overnight and the selection medium consisted of F-12K/Ham's, 10% fetal bovine serum (FBS), 100 μg/ml of Geneticin, and 10 μg/ml Blasticidin S HCl, and penicillin with streptomycin. Stably expressing clones were maintained in the same medium except that the Balsticidin S HCl concentration was reduced to 5 μg/ml.

### Electrophysiology

Whole-cell recordings of TRP channel currents were obtained from acutely trypsinized TRPM5-expressing HEK cells. The bath solution was Hank's Balanced Salt Solution, composed of (mM); 1.2 CaCl₂, 0.5 MgCl₂-6H₂O, 0.4 MgSO₄-7H2O, 5.3 KCl, 0.4 KH₂PO₄, 137.9 NaCl, 0.3 Na₂HPO₄-7H₂O, and 5.5 D-Glucose, with 20 mM HEPES (Invitrogen), pH 7.4 (NaOH). The internal pipette solution contained, in mM: 135 glutamic acid, 8 NaCl, 3 CaCl₂, 10 HEPES and 10 EGTA, pH 7.2 (CsOH) (Sigma). Calculated concentration of free calcium in internal solution was 77 nM (MaxChelator, Stanford University). Recording pipettes were pulled using a Flaming/Brown Micropipette Puller (Sutter Instruments), from fire-polished borosilicate glass, to approximately 2 MΩ. Voltage clamp recordings were obtained in whole cell mode using MultiClamp 700B amplifier and Digidata 1322A converter running on Clampex 9.2 software (Axon Instruments). Recordings were performed at room temperature. Series resistance was automatically compensated immediately after the break-in. Data were sampled at 5 kHz and filtered at 1 kHz. AITC was dissolved in bath solution and applied to the cells with a multi-barrel applicator (SF-72, Warner Instruments).

### FLIPR® Assay

For hTRPM5-293 or HEK293 assays, cells were seeded overnight in poly-D-lysine coated 384-well plates at 15,000 cells per well in 20 μl of media. For assays of hTrpM5-CHO-M1 assays cells were seeded overnight in tissue culture treated 384-well plates at 10,000 cells per well in 20 μl of media. The assay was performed using a fluorometric imaging plate reader (FLIPR-Tetra™, Molecular Devices, Sunnyvale, Calif.), using the excitation 510-545 nm and emission 565-625 nm filter sets. The cells were loaded with 20 μl/well of Membrane Potential Assay Kit RED dye (Molecular Devices), in a 37° C. and 5% CO₂ incubator for 1 hour. To measure intracellular calcium changes, the Calcium 3 dye (Molecular Devices) supplemented with 125 μM was used. The plates were equilibrated to room temperature for 15 minutes before the start of the assay. The compounds allyl isothiocyanate (AITC), cinnamaldehyde, EGTA, ionomycin, U73122, U73144 were purchased from Sigma-Aldrich (St. Louis, Mo.) and stocks prepared in DMSO. Samples were diluted in HBSS with 20 mM HEPES prior to the assay and 10 μl per well was added to the assay plate. The plates were read on the FLIPR® for a total of 3 minutes for a single addition assay and 6 minutes for a 2 addition assay. For single addition assays, baseline fluorescence was obtained on the FLIPR® for 10 seconds followed by addition of each sample by the FLIPR® and read for an additional 2 minutes and 50 seconds. For 2 addition assays, baseline fluorescence was obtained on the FLIPR® for 10 seconds followed by addition of the first sample (e.g. inhibitors, EGTA) by the FLIPR®, read for 2 minutes and 50 seconds, then followed by the addition of AITC and read for another 3 minutes. For ΔRFU measurements, the baseline fluorescence signal (RFUmin) was subtracted from the peak fluorescence signal (RFUmax) at each compound concentration (RFUmax—RFUmin). ΔRFU values for individual concentrations were measured in triplicate, and s.d. reported as error bars.

### Results

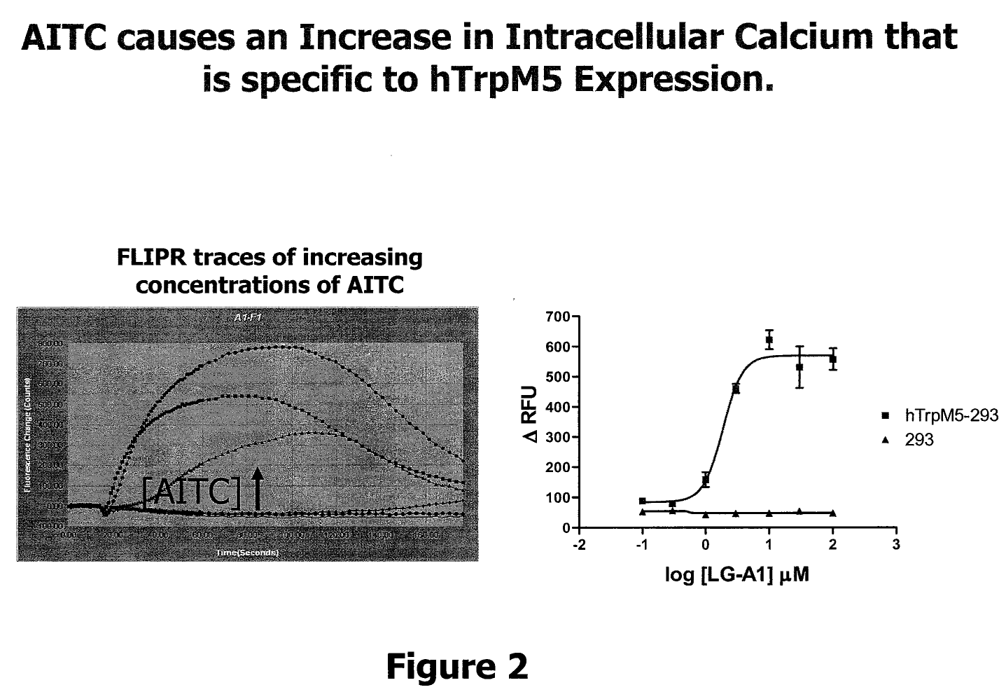

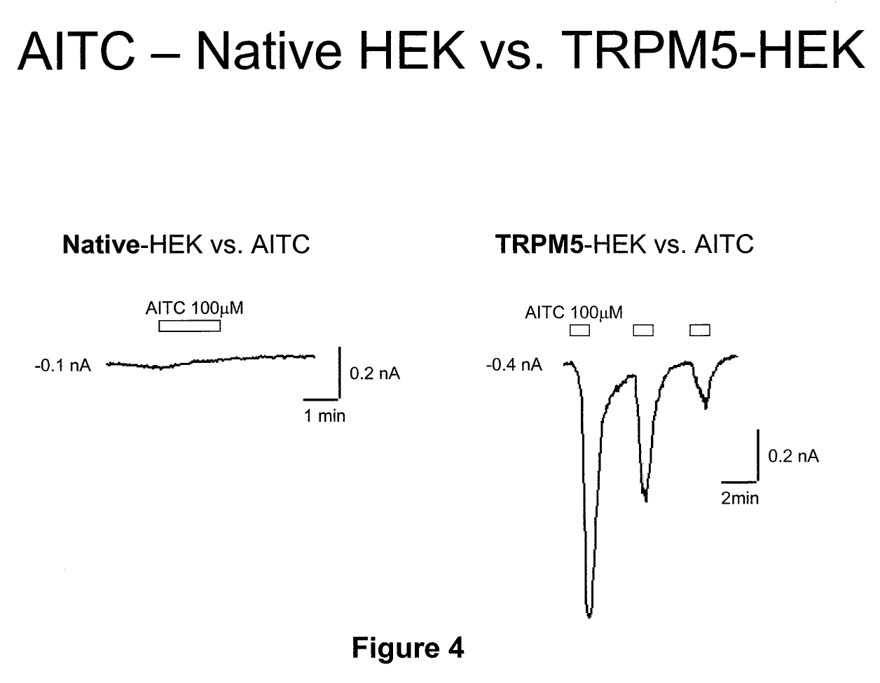

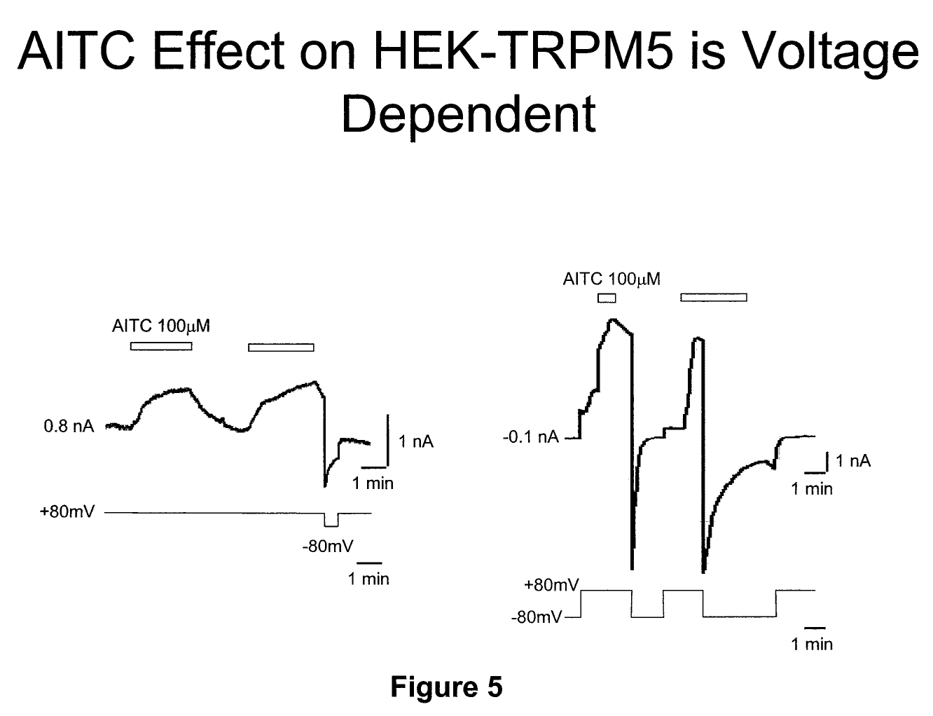

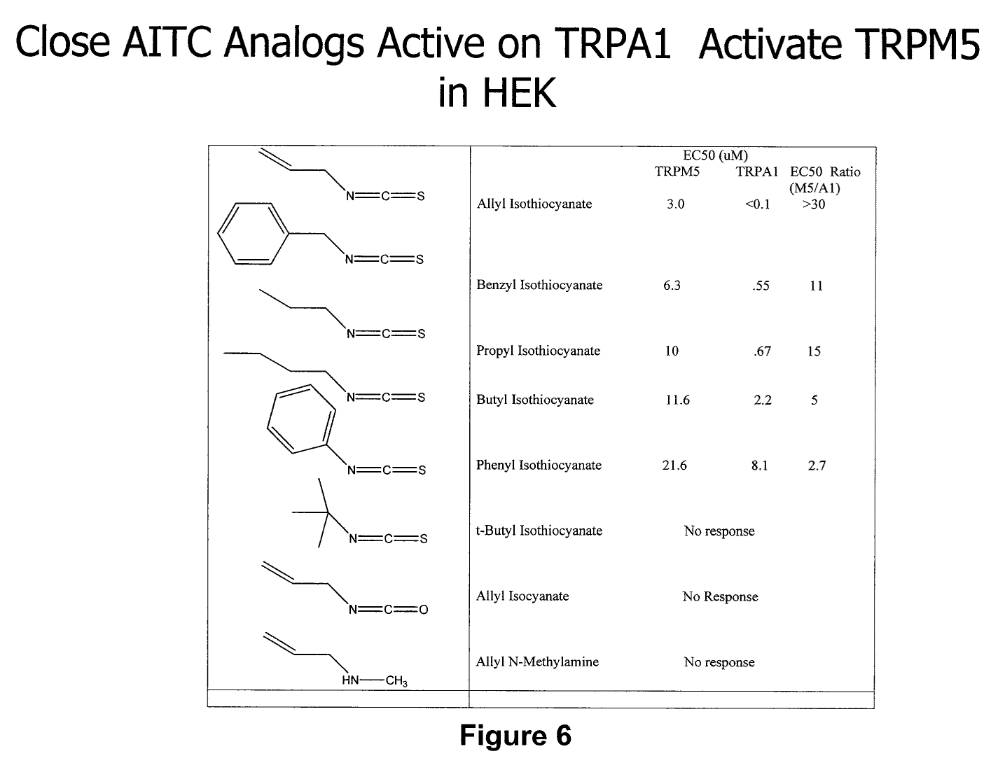

HEK 293 cells expressing human TRPM5 (TRPM5-293) were incubated with AITC, a selective TRPA1 agonist. AITC caused a strong membrane potential response in the TRPM5-expressing cells (FIG. 1). In addition, AITC caused an increase in intracellular calcium levels that was specific to TRPM5 expression because there was no response in untransfected cells (FIG. 2). Importantly, AITC does not activate TRPM5 when it is expressed in CHO cells (FIG. 3). This data indicates that AITC does not inherently activate TRPM5, but rather acts through the cooperativity mechanism between TRPA1 and TRPM5. Desensitization of the current is delayed at positive membrane potentials (FIGS. 4 and 5). This suggests that the activation of current by AITC goes through TRPA1 and not TRPM5, implying that TRPA1 can serve as a trigger or amplifier for TRPM5 activation. The AITC effect was not limited to AITC however, as AITC analogs that have been shown to activate TRPA1 were also shown to activate TRPM5 in the TRPM5-HEK293 cells (FIG. 6).

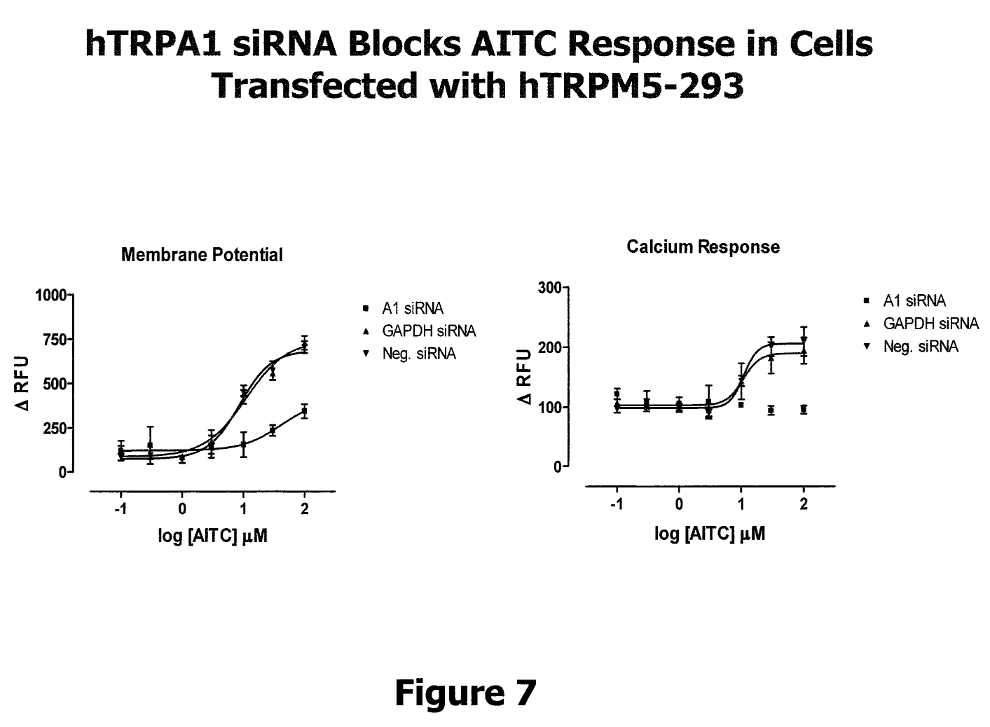

AITC activation of TRPM5 through TRPA1 was also confirmed by suppressing TRPA1 expression. hTRPM5-293 cells were transfected with 7.5 μl each of Ambion Precision hTRPA1-targeted siRNA, GAPDH-targeted siRNA, or scrambled negative siRNA (Ambion, Austin, Tex.). Cells were transfected in OPTI-MEM media (Invitrogen) using the transfection reagent siPORT Amine (Ambion), following manufacturer's instructions for optimization of reagent to siRNA ratios. Cells were plated in six-well plates at a density of 300,000 cells/well (HEK) or 150,000 cells/well (CHO) one day prior to transfection, and were used for experiments at least 24 hours after transfection. As shown in FIG. 7, siRNA specific for TRPA1 blocked the expression of both the membrane potential and calcium response in TRPM5-293 stable cell lines.

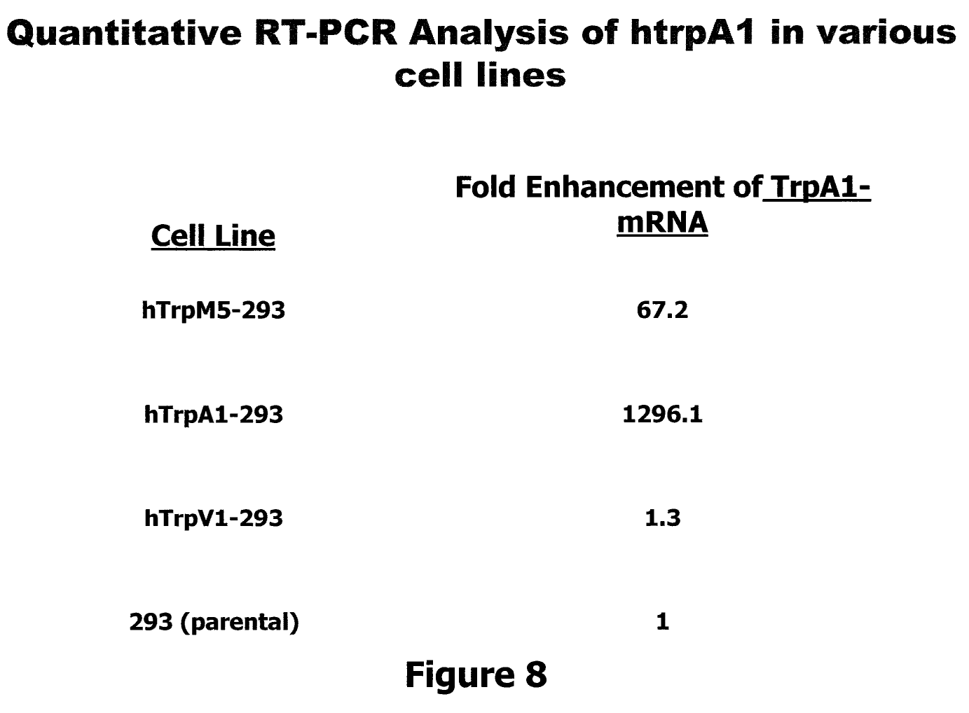

Interestingly, expression of TRPM5 in the TRPM5-293 cells actually resulted in enhancement of TRPA1 mRNA levels. RNA was isolated from human TRPM5-293, human TRPA1-293, human TRPM8-293, mouse TRPM5-293 stable cell lines as well as naive HEK 293 cells using a RNeasy Mini Kit (Qiagen). Purified RNAs were DNased by DNase I Amplification Grade (Invitrogen). cDNA was synthesized from RNA by SuperScript First Strand Synthesis System for RT-PCR (Invitrogen). Real Time PCR was performed using TaqMan Fast Universal PCR Master Mix (Applied Biosystems) in ABI 7500 Fast RT-PCR System by using specific primers for human TRPA1 (Applied Biosystems) and human GAPDH (Applied Biosystems) and duplexing under the following conditions: 1 cycle at 95° C. for 20 s; 40 cycles at 95° C. for 3 s, 60° C. for 30 s. The data was analyzed by normalizing to GAPDH. Naive HEK 293 CT values were taken as the negative control. As shown in FIG. 8, TRPM5-HEK293 stable cell lines demonstrated a 67-fold enhancement in TRPA1 MRNA levels when compared to control cell lines.

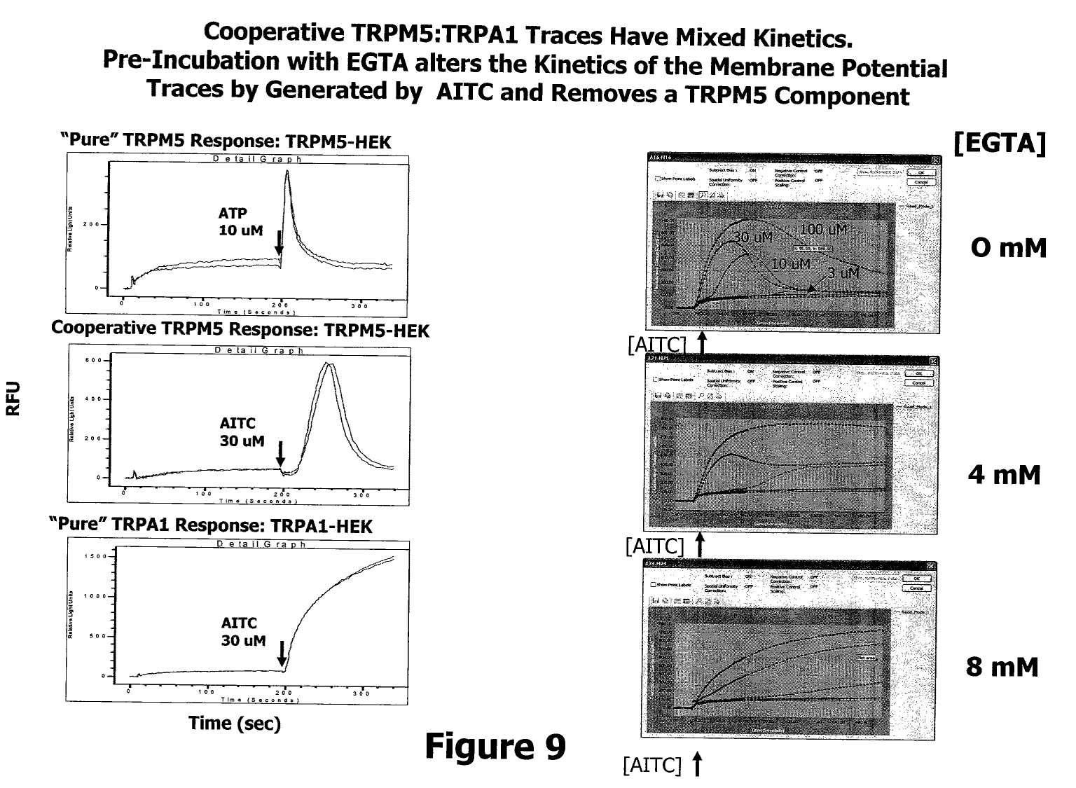

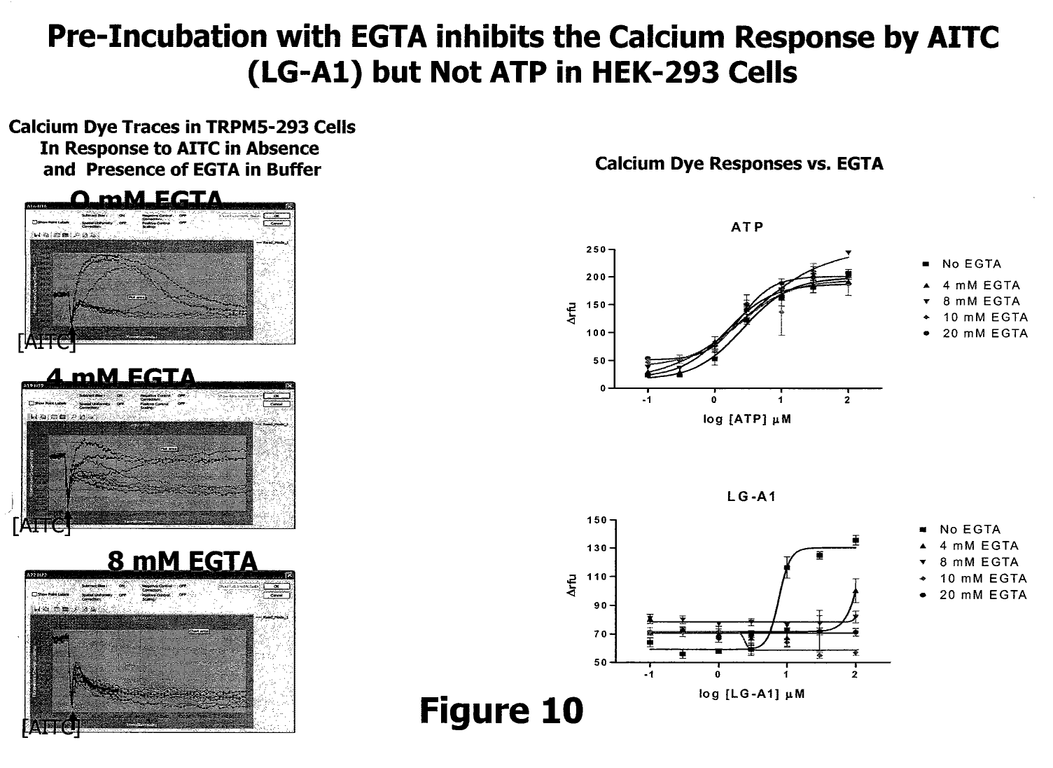

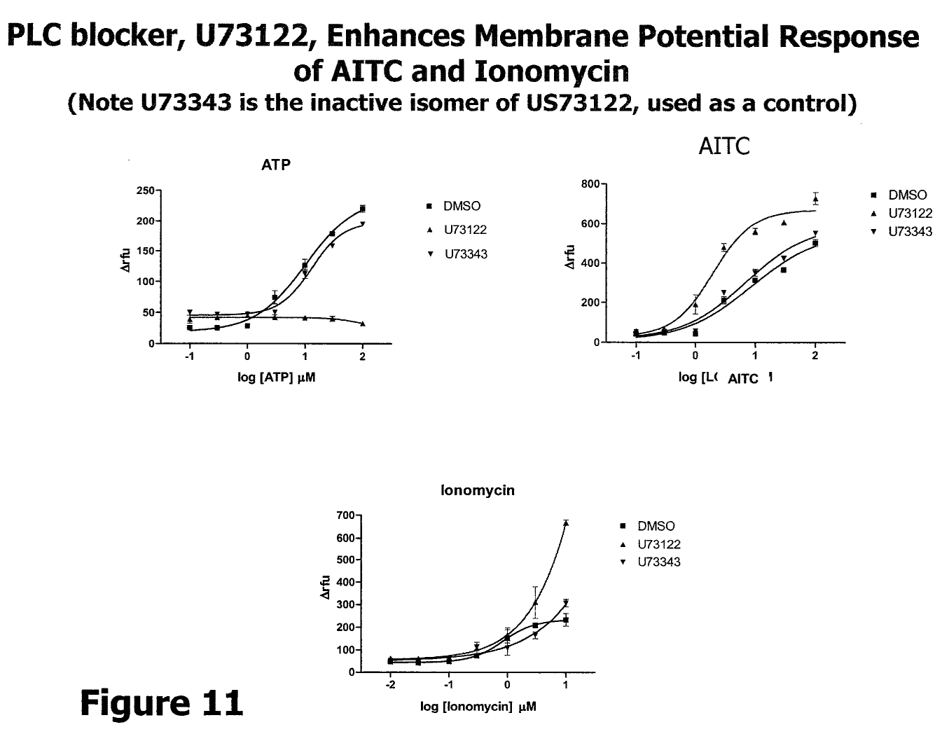

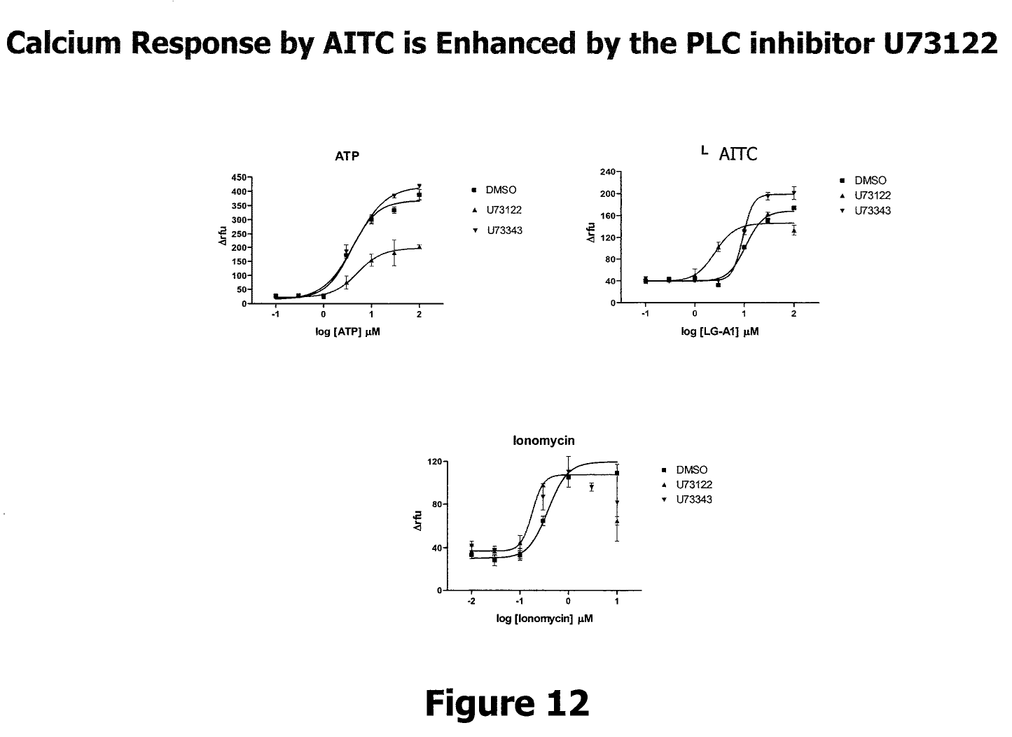

TRPA1 activation by AITC in TRPM5-293 cells also triggers calcium influx. Pre-incubation of TRPM5-293 cells with the chelating agent EGTA altered the membrane potential (FIG. 9) and inhibited the calcium response (FIGS. 10 and 11) in response to AITC. As shown in FIGS. 9 and 10, increasing concentrations of EGTA significantly altered membrane potential responses as well as blocking the AITC-induced calcium response. This demonstrates that the AITC response is dependent on extracellular calcium. Importantly however, the response was not dependent on effectors downstream of phopholipase C. When TRPM5-293 cells were exposed to the PLC inhibitor U73122, both the membrane potential and calcium responses were enhanced in the cells following exposure to AITC (FIGS. 11 and 12).

### Example 2

### Modulators of TRPM5 Alter Responses to TRPA1 Agonists

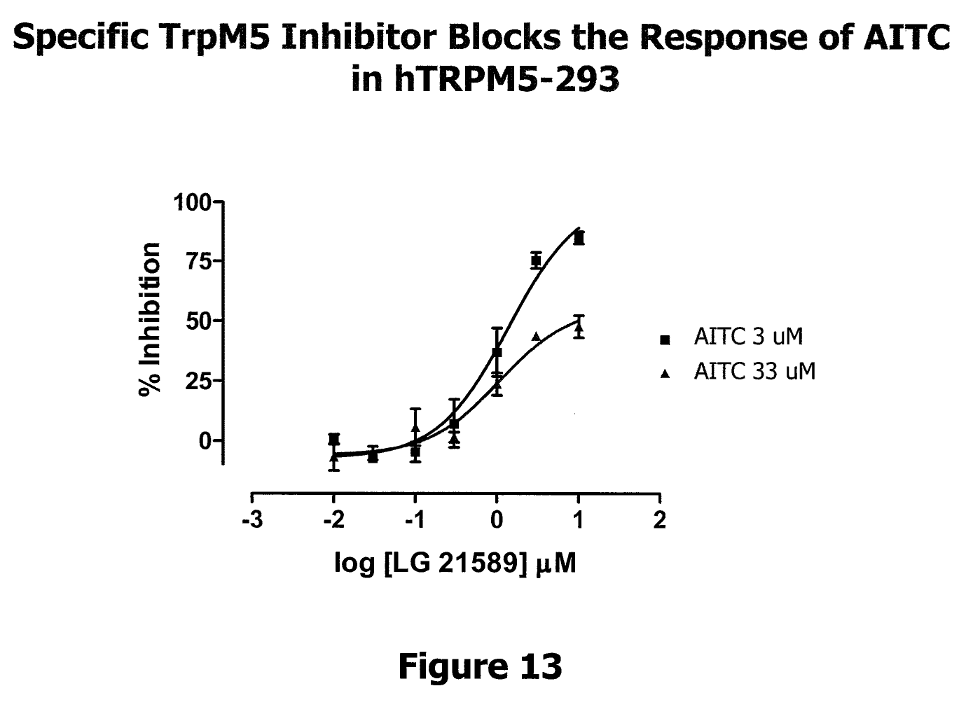

The TRPM5-specific inhibitor LG 21589 was tested for its ability to block the AITC response in TRPM5-293 cells. As shown in FIG. 13, LG 21589 was able to block the AITC response in the TRPM5-expressing cells in a dose-dependent manner, as cells exposed to 33 μM AITC were inhibited approximately 45% compared to those cells exposed to 3 μM AITC.

### Example 3

### Inhibitors of TRPA1 Activity Inhibit TRPM5 Activity

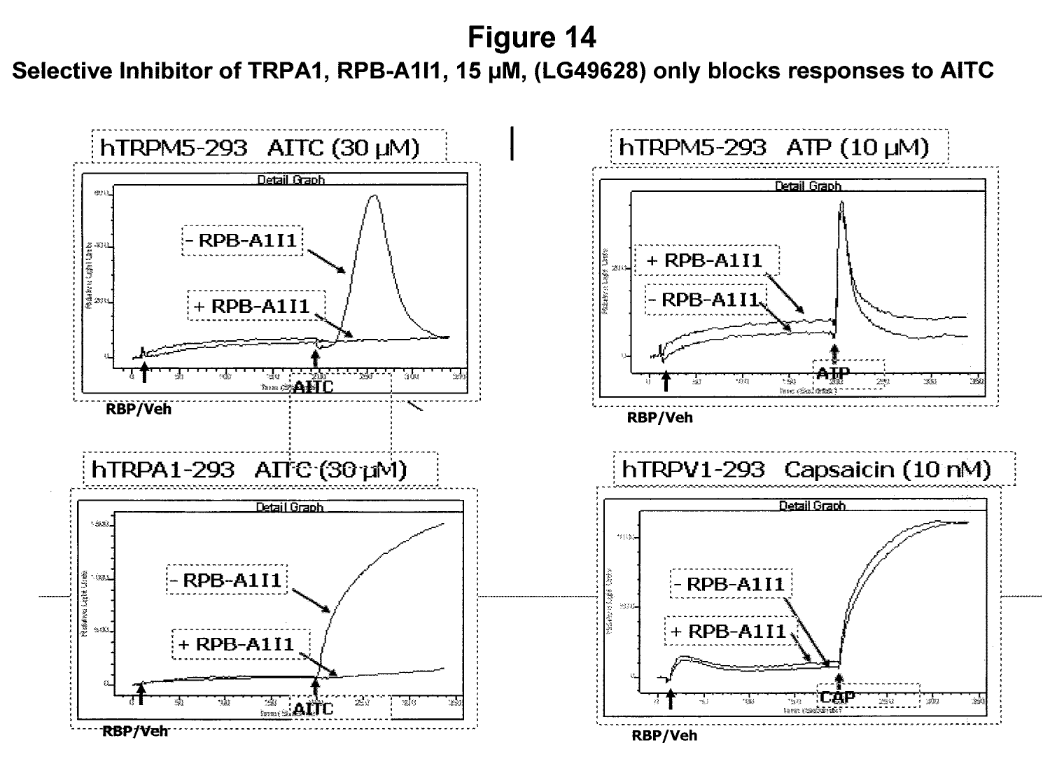

The TRPA1-specific inhibitor RPB-A1|1 (LG49628) was tested for its ability to block the AITC response in TRPM5-293 cells. As shown in FIG. 14, 15 μM RPB-A1|1 was able to block the AITC response (30 μM) in the TRPM5-expressing cells in a dose-dependent manner. The IC₅₀ was approximately 5-7 μM (data not shown). RPB-A1|1 did not affect either ATP-induced responses in TPRM5-293 cells, or capsaicin responses in TRPV1-293 cells.

### Example 4

### TRPM5 is Expressed in Human and Mouse Neuronal Tissue

TRPM5 was found to be expressed in tissue associated with TRPA1, namely neuronal tissue. Co-expression of TRPM5 and TRPA1 in human and mouse dorsal root ganglia confirms that TRPM5 modulators can also modulate the activity of TRPA1.

### RT-PCR Method

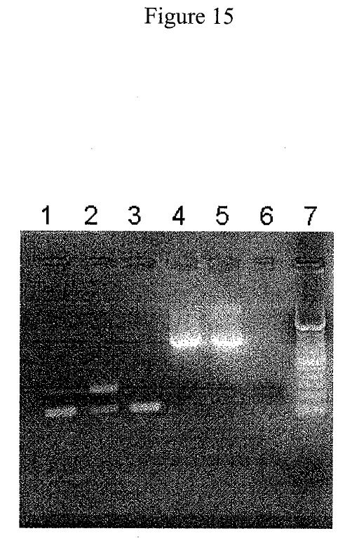

RNA was isolated from mouse dorsal root ganglia (DRG) cells using a RNeasy Mini Kit (Qiagen, Valencia, Calif.). Purified RNAs were digested by DNase I Amplification Grade (Invitrogen, Carlsbad, Calif.). cDNA was synthesized from RNA using SuperScript First Strand Synthesis System for RT-PCR (Invitrogen). Real Time PCR was performed using TaqMan Fast Universal PCR Master Mix (Applied Biosystems, Foster City, Calif.) in ABI 7500 Fast RT-PCR System by using specific primers for human TRPA1 (Applied Biosystems) and human GAPDH (Applied Biosystems) and duplexing under the following conditions: 1 cycle at 95° C. for 20 seconds; 40 cycles at 95° C. for 3 seconds, 60° C. for 30 seconds. Human dorsal ganglion RNA was purchased from Clontech (Mountain View, Calif.) (catalog #636150) and mouse dorsal ganglion RNA was also isolated from mixed C57BL/6 and 129. RT-PCR was done in duplicates and was duplexed against the house keeping gene glyceraldehyde 3-phosphate dehydrogenase (GAPDH). The data was analyzed by normalizing to GAPDH. Similar results were obtained using standard PCR techniques on cDNA from mouse DRG cells (FIG. 15).

| Tissue | GAPDH | Trpm5 | TrpA1 |
|---|---|---|---|
| Human Dorsal ganglion | 19.908 | 38.982 | |
| Human Dorsal ganglion | 19.593 | | 34.313 |
| Mouse Dorsal ganglion | 24.791 | 36.533 | |
| Mouse Dorsal ganglion | 24.865 | | 33.713 |

### Imaging

Six-week-old mixed C57BL/6-129 mice (purchased from Deltagen, San Mateo, Calif.) were euthanized under CO₂ and decapitated. Briefly, C57BL/6-129 mice were generated using a C57BL/6 blastocyst strain and a 129 ES strain. Laminectomy was performed and 5-10 DRGs were collected from all spinal levels. DRGs were dissociated enzymatically and mechanically. Dissociated DRG neurons were plated onto poly-L-lysine-coated glass-bottom of 35mm culturing dishes. Cells were used fresh or grown for up to three weeks in a 37° C. and 5% CO₂ incubator. Culture medium consisted of Dulbecco's Modified Eagle Medium (DMEM), 10% fetal bovine serum (FBS), and penicillin with streptomycin.

DRG cells were viewed through a 40× Plan Fluor magnification objective (Nikon, Japan) using a TE2000-S inverted microscope (Nikon). Images were acquired with a CoolSnap HQ2 camera (Photometrics, Tucson, Ariz.). A xenon lamp (175W; Intracellular Imaging Inc., Cincinnati, Ohio) controlled by the Lambda 10 shutter controller (Sutter Instruments) was used to excite cells at 488 nm. LacZ (β-galactosidase) staining was performed with 1 mM Fluorescin Digalactoside (FDG) (Molecular Probes, Invitrogen, Carlsbad, Calif.) dissolved in hypotonic (150 mOsm) HBSS solution for 1 min at 37° C. After staining the dish was kept on ice till imaging.

The bath solution was HBSS (Invitrogen), composed of (mM); 1.2 CaCl₂, 0.5 MgCl₂·6H2O, 0.4 MgSO₄·7H₂O, 5.3 KCl, 0.4 KH₂PO4, 137.9 NaCl, 0.3 Na₂HPO₄·7H₂O, and 5.5 d-Glucose, supplemented with 20 mM HEPES (Invitrogen), pH 7.4 (NaOH).

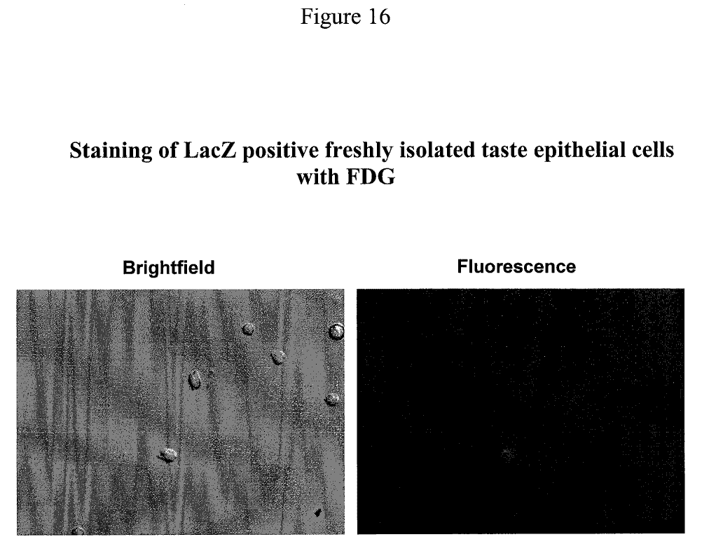

FIG. 16 shows brightfield (left) and fluorescent (right) images captured of freshly isolated taste epithelial cells obtained from TRPM5-LacZ mice. These mice express TRPM5 under control of a LacZ promoter. Thus, TRPM5 expression is associated with expression of β-galactosidase in these cells. Cells were loaded with FDG in hypotonic HBSS for 1 minute at 37° C. and then kept on ice until imaged. FIG. 16 shows that one out of seven cells in the field of view stained positive for LacZ.

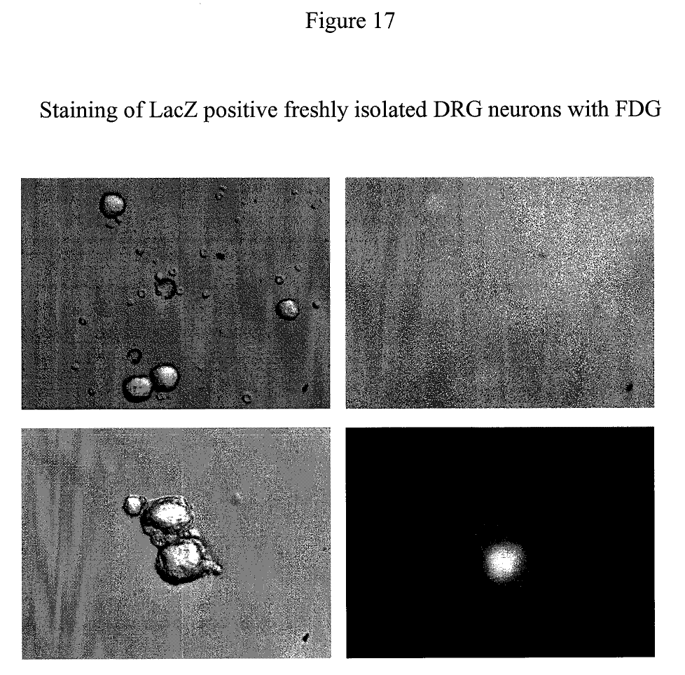

FIG. 17 shows brightfield (left) and fluorescent (right) image of freshly isolated DRG neurons obtained from TRPM5-LacZ mouse. Neurons were loaded with FDG in hypotonic HBSS for 1 minute at 37° C. and then kept on ice until imaged.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. All publications, patents and patent applications cited herein are incorporated by reference in their entirety into the disclosure.

What is claimed is:

1. A method of modulating TRPA1-mediated processes comprising administering a modulator of TRPM5 activity.

2. The method of claim 1, wherein said TRPA1 and TRPM5 are human.

3. The method of claim 1, wherein said administration is done in vivo.

4. The method of claim 1, wherein TRPA1 is present in a TRPM5-expressing cell or cultured neuron.

5. The method of claim 1, wherein the processes are selected from the group consisting of pain, mechanosensation and taste.

6. The method of claim 1, wherein said modulator causes an increase in TRPM5 activity.

7. The method of claim 1, wherein said modulator causes a decrease in TRPM5 activity.

8. A method of inhibiting TRPA1-mediated pain signaling by inhibiting TRPA1 activity, comprising administering to a subject in need an inhibitor of TRPM5 expression.

9. The method of claim 8, wherein TRPA1 is present in a TRPM5-expressing cell or cultured neuron.

10. The method of claim 8, wherein said TRPA1 and TRPM5 are human.

11. The method of claim 8, wherein TRPM5 expression is inhibited using RNA interference, antisense oligonucleotides, ribozymes, aptamers or antibodies.

12. The method of claim 8, wherein said TRPA1 activity is measured by measuring calcium influx in said TRPA1-expressing cell.

13. The method of claim 8, wherein said TRPA1 activity is measured by measuring the enzymatic activity of the phospholipase C polypeptide.

14. The method of claim 13, wherein said enzymatic activity is the breakdown of phosphatidylinositol-4,5-bisphospate (PIP2) into diacylglycerol (DAG) and inositol triphosphate (IP3).

15. The method of claim 8, wherein said pain is selected from the group consisting of acute, chronic, neuropathic and nociceptive.

16. A method of inhibiting TRPA1-mediated signaling comprising administering an inhibitor of TRPM5.

17. A method of increasing TRPA1 expression in a cell comprising expressing TRPM5 in said cell.

18. The method of claim 17, wherein said TRPM5 expression is at a greater level than expressed in wild-type cells.

19. The method of claim 17, wherein said TRPM5 is exogenously added to said TRPA1 expressing cell.

20. A method of amplifying TRPM5 activation comprising administering an activator of TRPA1 activity.

21. The method of claim 20, wherein said activator of TRPA1 is selected from the group consisting of cinnamaldehyde, eugenol, gingerol, methyl salicylate, AITC and allicin.

22. A method of inhibiting TRPM5 activity comprising administering an inhibitor of TRPA1 activity.

23. A method for identifying an agent that inhibits TRPA1 activity through TRPM5 signaling comprising:
(a) contacting a cell that expresses both TRPA1 and TRPM5 with an agent;
(b) measuring the activity of TRPM5,
(c) contacting another cell that expresses both TRPA1 and TRPM5 with the same agent as in step (a);
(d) measuring the activity of TRPA1;
(e) identifying an agent that decreases both TRPM5 and TRPA1 activity.

24. The method of claim 23, wherein said TRPA1 and TRPM5 are human.

25. The method of claim 23, wherein said TRPM5 activity is measured by measuring the membrane potential of said cell.

26. The method of claim 23, wherein said TRPA1 activity is measured by measuring calcium influx in said cell.

27. The method of claim 23, wherein said TRPA1 activity is measured by measuring the enzymatic activity of phospholipase C.

28. The method of claim 27, wherein said enzymatic activity is the breakdown of phosphatidylinositol-4,5-bisphospate (PIP2) into diacylglycerol (DAG) and inositol triphosphate (IP3).

29. A method of modulating calcium-activated ion channel activity comprising administering a modulator of TRPA1 activity to a cell.

30. The method of claim 29, wherein said calcium-activated ion channel is TRPM5.

31. The method of claim 29, wherein said modulator of TRPA1 activity is selected from the group consisting of cinnamaldehyde, eugenol, gingerol, methyl salicylate, AITC and allicin.

32. The method of claim 29, wherein said calcium-activated ion channel activity is measured by measuring the membrane potential of said cell.

33. The method of claim 29, wherein said calcium-activated ion channel activity is measured by measuring calcium influx in said cell.
